(19) United States
(12) Patent Application Publication
(10) Pub. No.: US 2014/0194806 A1
(43) Pub. Date: Jul. 10, 2014

(54) EXTERNAL ANCHORING CONFIGURATIONS FOR MODULAR GASTROINTESTINAL PROSTHESES

(71) Applicant: MetaModix, Inc., Plymouth, MN (US)

(72) Inventors: Kedar R. Belhe, Minnetonka, MN (US); Paul Thompson, Minnetonka, MN (US); Alexander Garfov, Eden Prairie, MN (US); Werner Schwarz, Ruhpolding (DE)

(21) Appl. No.: 14/203,190

(22) Filed: Mar. 10, 2014

Related U.S. Application Data
(63) Continuation-in-part of application No. 13/568,101, filed on Aug. 6, 2012, now Pat. No. 8,702,642.
Application No. 13/568,101 is a continuation-in-part of application No. 12/833,605, filed on Jul. 9, 2010, now Pat. No. 8,282,598.
(60) Provisional application No. 61/270,588, filed on Jul. 10, 2009.

(51) Int. Cl. A61F 5/00; A61M 27/00; A61B 17/132

(52) U.S. Cl. CPC A61F 5/0076; A61B 17/1322; A61M 27/002
USPC 604/8; 606/151

(57) ABSTRACT

Components may be used separately or in combination to create anchoring systems for intra-luminal implants for the treatment of metabolic disorders such as obesity and diabetes. Various systems include an external component adapted for deployment around a portion of the gastrointestinal tract (e.g., the duodenum) and an internal component adapted for implantation within the gastrointestinal tract. Various systems use anchoring means that are based on mechanical interference, elasticity, spring force, shape memory transformation, magnetic attraction, repulsion and/or levitation. Various embodiments rely on longitudinal anchoring of the implants with minimal force against tissue.

### CROSS-REFERENCE TO RELATED APPLICATION

This application is a Continuation-In-Part of U.S. patent application Ser. No. 13/568,101, filed Aug. 6, 2012, entitled “External Anchoring Configurations for Modular Gastrointestinal Prostheses,” which is a Continuation of U.S. patent application Ser. No. 12/833,605, filed Jul. 9, 2010, entitled “External Anchoring Configurations for Modular Gastrointestinal Prostheses,” and having U.S. Pat. No. 8,282,598, which claims the benefit under 35 U.S.C. §119(e) to U.S. Provisional Application 61/270,588, filed Jul. 10, 2009, all of which are incorporated herein by reference in their entirety.

### TECHNICAL FIELD

This invention generally relates to implants placed within gastrointestinal systems, including the esophagus, the stomach and the intestines. In particular, it relates to implant systems having components implantable and removable using laparoscopic and endoscopic techniques for treatment of obesity, diabetes, reflux, and other gastrointestinal conditions.

### BACKGROUND

Bariatric surgery procedures, such as sleeve gastrectomy, the Rouen-Y gastric bypass (RYGB) and the bileo-pancreatic diversion (BPD), modify food intake and/or absorption within the gastrointestinal system to effect weight loss in obese patients. These procedures affect metabolic processes within the gastrointestinal system by either short-circuiting certain natural pathways or creating different interaction between the consumed food, the digestive tract, its secretions and the neuro-hormonal system regulating food intake and metabolism. In the last few years, there has been a growing clinical consensus that obese diabetic patients who undergo bariatric surgery see a remarkable resolution of their Type-2 Diabetes Mellitus (T2DM) soon after the procedure. The remarkable resolution of diabetes after RYGB and BPD typically occurs too fast to be accounted for by weight loss alone, suggesting that there may be a direct impact on glucose homeostasis. The mechanism of this resolution of T2DM is not well understood, and it is quite likely that multiple mechanisms are involved.

One of the drawbacks of bariatric surgical procedures is that they require fairly invasive surgery with potentially serious complications and long patient recovery periods. In recent years, there is an increasing amount of ongoing effort to develop minimally invasive procedures to mimic the effects of bariatric surgery using minimally invasive procedures. One such procedure involves the use of gastrointestinal implants that modify transport and absorption of food and organ secretions. For example, U.S. Pat. No. 7,476,256 describes an implant having a tubular sleeve with an anchor having barbs. Some particular exemplary devices or systems are included in a patent and a patent application assigned to the same assignee as the present invention, which are U.S. Pat. No. 8,211,186; and US Publication No. 2012/0253259, and are herein incorporated by reference. While these implants may be delivered endoscopically, the implants offer the physician limited flexibility and are not readily removable or replaceable, since the entire implant is subject to tissue in-growth after implantation. Moreover, stents with active fixation means, such as barbs that penetrate into the surrounding tissue, may potentially cause tissue necrosis and erosion of the implants through the tissue, which can lead to serious complications, such as systemic infection. Also, due to the intermittent peristaltic motion within the digestive tract, implants such as stents have a tendency to migrate.

### SUMMARY

According to various embodiments, the present invention is a gastrointestinal implant system for treating metabolic disorders, such as diabetes and obesity. The system includes a tubular implant adapted for placement within at least a portion of the duodenum, the tubular implant having a securing feature and an external band configured for implantation around at least one of a pylorus, and a duodenum, the external band having a coupling feature for removably engaging and coupling with the securing feature of the internal tubular implant without penetrating the duodenum or pylorus, such that the therapeutic implant resists migration within the gastrointestinal tract, wherein the external band has an inner diameter generally equal to a corresponding outer diameter of the duodenum or pylorus, and the securing feature and coupling feature are configured such that the tubular implant is releasably coupled to the external band to facilitate removal of the tubular implant.

According to various embodiments, the present invention is a modular gastrointestinal implant system for treating metabolic conditions, such as diabetes and obesity. The system includes an external implant configured for affixing around at least a portion of a duodenum or pylorus, the external implant having a docking feature, and a therapeutic implant adapted for placement within a gastrointestinal tract, the therapeutic implant having a securing feature adapted to removably couple with the docking feature without penetrating the gastrointestinal tract, such that the therapeutic implant resists migration within the gastrointestinal tract, wherein the external band has a diameter generally equal to the diameter of the duodenum or pylorus.

According to other exemplary embodiments, the present invention is a method of treating metabolic conditions, such as diabetes and obesity. The method includes placing an external implant around at least a portion of the duodenum, the external implant having a docking feature and the external implant having an inner diameter generally equal to an outer diameter of the corresponding portion of the duodenum, implanting, using a minimally-invasive technique, an internal tubular implant having a securing feature to a location within the duodenum corresponding to the location of the external implant, and causing the securing feature to removably couple with the docking feature without penetrating the duodenum.

According to various disclosed embodiments, systems for anchoring intra-luminal implants within hollow body organs (e.g., the gastrointestinal organs) include an external fixation mechanism that can be delivered to an external surface of the organ (e.g., by laparoscopic techniques) and an intra-luminal implant configured to engage with the external fixation means, without the need for excessive radial force on the organ and without penetrating the tissue. According to various embodiments, the fixation mechanisms operate using techniques such as shape modification of the organ to capture the implant longitudinally or magnetic attraction, repulsion or levitation of the implant. Various embodiments of the present invention are useful for treating metabolic conditions, including for example, diabetes and obesity.

The present invention according to various embodiments is a modular system for creating internal bypass of food and organ secretions within the gastrointestinal tract that includes low-profile implants that are affixed around the stomach, the esophagus, the intestine or externally around junctions of these organs, and gastrointestinal implants that permit internal by-pass of food and organ secretions from one site within the gastrointestinal tract to other sites within the gastrointestinal tract that have complementary design features to the external implant that enables secure placement within the gastrointestinal tract.

The present invention according to various embodiments is a modular system for creating a completely reversible internal bypass of food and organ secretions within the gastrointestinal tract that includes low-profile implants that are affixed around the stomach, the esophagus, the intestine or externally around junctions of these organs and which enable secure attachment of other implants within the gastrointestinal tract, and gastrointestinal implants that permit internal by-pass of food and organ secretions from one site within the gastrointestinal tract to other sites within the gastrointestinal tract that have complementary design features to the external implant that enables secure placement within the gastrointestinal tract.

The present invention according to various embodiments is a modular system for treating gastro-esophageal reflux disease (GERD) that includes low-profile implants that are affixed around the stomach, the esophagus, the intestine or externally around junctions of these organs and which enable secure attachment of other implants within the gastrointestinal tract, and an internal tubular implant of a design that normally permits only one-way passage of food from the esophagus to the stomach and that can be secured within the gastrointestinal tract by the external low-profile implant.

The present invention according to various embodiments is a method for creating a reversible treatment for metabolic disorders, such as diabetes and obesity, and for the treatment of gastro-esophageal reflux disease (GERD), including placing low-profile implants that can be affixed around the stomach, the esophagus, the intestine or externally around junctions of these organs and which enable secure attachment of other implants within the gastrointestinal tract, and placing other gastrointestinal implants that permit internal by-pass of food and organ secretions from one site within the gastrointestinal tract to other sites within the gastrointestinal tract, which do not directly anchor to the tissue but are securely held by the external implant so that the procedure can be reversed easily.

The present invention according to various embodiments is a method of treating metabolic disorders, such as obesity and diabetes, by placing a permanent band like structure around the esophagus, the stomach, the intestine or externally around junctions of these organs, and endoscopically placing a long tubular sleeve within the GI tract with expandable elements at its ends, those expandable elements having design functionality that enables it to be reversibly secured in position by the external band.

The present invention according to various embodiments is a method for creating a gastrointestinal bypass, the method including delivering a band like structure at appropriate locations around the gastrointestinal tract, such as the esophagus, the stomach, the duodenal bulb, the pyloric junction, the gastro-esophageal junction, etc.; and delivering a tubular sleeve with expandable ring shaped elements at its ends, those rings having outward indentations that enable it to be reversibly secured in position by the external band.

According to various embodiments, as a second mode of anchoring, stabilizing, or preventing migration, the external band is coupled to an anatomical feature (e.g., a ligament) external to the tissue of the esophagus, stomach, pylorus, or intestine. In some embodiments, the external band is intertwined with, interlocked with or threaded between the anatomical feature and the tissue. According to one exemplary embodiment, the external band is coupled to the hepatoduodenal ligament. This second mode of anchoring enables the use of external bands that do not rely on excessive compressive force to keep the implant in place, since excessive compressive forces can cause tissue necrosis and erosion.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a sectional view of a portion of the digestive tract in the body showing an external band implanted around the outside diameter of the duodenal bulb and a tubular implant (sleeve) implanted on the inside surface of the duodenal bulb and anchored magnetically through the duodenal bulb tissue to the external band. The tubular implant extends into the duodenum to the ligament of Treitz.

FIGS. 2-7 show various embodiments of an external band that may be used as an external anchoring device to secure an internal tubular implant.

FIG. 8 shows a tubular implant that can be used to bypass the stomach, duodenum or other intestinal lumen.

FIG. 9 is a schematic view showing a trocar and cannula operable to access the implant location of the duodenal bulb using laparoscopic techniques.

FIG. 10 is a schematic view showing a cannula inserted to access the implant location of the duodenal bulb, and an external band implanted around the duodenal bulb.

FIG. 11 shows an exemplary endoscope used for diagnostic and therapeutic procedures in the gastrointestinal (GI) tract.

FIG. 12 is a sectional view of a portion of the digestive tract in the body, with an endoscope passing through the esophagus into the stomach, and the end of the scope is positioned to allow viewing of the pylorus.

FIG. 13A shows an over-the-wire sizing balloon that can be used to measure the diameter of the pylorus, duodenal bulb, esophagus, pyloric antrum or other lumen in the GI tract.

FIG. 13B shows a monorail sizing balloon that can be used to measure the diameter of the pylorus, duodenal bulb, esophagus, pyloric antrum or other lumen in the GI tract.

FIG. 14 is a sectional view of a portion of the digestive tract in the body, with an endoscope inserted into the GI tract up to the pylorus and a sizing balloon inserted through the working channel and into the area of the duodenal bulb. The balloon is inflated to measure the diameter of the duodenal bulb.

FIG. 15 shows the endoscope and delivery catheter advanced through the external anchoring device into the duodenum to the ligament of Treitz.

FIG. 16 shows the endoscope and delivery catheter advanced through the external anchoring device into the duodenum to the ligament of Treitz. The outer sheath of the delivery catheter is retracted to partially expose the tubular implant.

FIG. 17 shows the endoscope and delivery catheter advanced through the external anchoring device into the duodenum to the ligament of Treitz. The outer sheath of the delivery catheter is retracted to partially expose the tubular implant. A balloon catheter is inserted through the working channel of the endoscope to the area of the partially exposed tubular implant. The balloon is inflated to temporarily secure the tubular implant to the duodenum.

FIG. 18 shows the system of FIG. 17, where the outer sheath is retracted further to unsheath the tubular implant up to the duodenal bulb.

FIG. 19 shows the system of FIG. 18, where the endoscope has been withdrawn to the duodenal bulb. The balloon on the balloon catheter is then deflated and the balloon catheter is withdrawn to the duodenal bulb. The balloon is then re-inflated to open up and secure the proximal end of the tubular implant to the inside diameter of the docking element.

FIG. 20 shows an alternative device and method for deploying the proximal end of the tubular element.

FIG. 21 is a sectional view of a portion of the digestive tract in the body. An external anchoring device is implanted around the stomach a couple of inches below the gastro-esophageal junction. The external anchoring device can serve as a restrictive means and can form a pouch-like restrictive segment at the top of the stomach. An internal tubular implant is implanted from the external anchoring device at the stomach to the ligament of Treitz.

FIG. 22 is a sectional view of a portion of the digestive tract in the body. An external anchoring device is implanted around the esophagus at gastro-esophageal junction. An internal tubular implant is implanted from the external anchoring device at the gastro-esophageal junction to the ligament of Treitz.

FIG. 23 is a sectional view of a portion of the digestive tract in the body. An external anchoring device is implanted around the pylorus. An internal tubular implant is implanted from the external anchoring device at the pylorus to the ligament of Treitz.

FIG. 24 is a sectional view of a portion of the digestive tract in the body. An external anchoring device is implanted around the stomach antrum. An internal tubular implant is implanted from the external anchoring device at the stomach antrum to the ligament of Treitz.

FIG. 25 is a sectional view of a portion of the digestive tract in the body. An external anchoring device is implanted around the duodenal bulb. An internal tubular implant is implanted from the duodenal bulb to the ligament of Treitz.

FIG. 26 is a sectional view of a portion of the digestive tract in the body. An external anchoring device is implanted around the duodenal bulb. An internal tubular implant is implanted from the duodenal bulb to the ligament of Treitz. The internal tubular implant uses a stent which has magnets integrated into it which attract to the magnets on the external band.

FIG. 27 is a sectional view of a portion of the digestive tract in the body. An external anchoring device is implanted around the duodenal bulb. An internal tubular implant is implanted from the duodenal bulb to the ligament of Treitz. The internal tubular implant uses a stent which has magnets integrated into it. The magnets on the external band and magnets on the internal band repel each other and limit movement or dislodgement of the internal tubular implant.

FIGS. 28-31 show sectional views of various embodiments of an external anchoring device implanted around the duodenal bulb. An internal tubular implant is implanted from the duodenal bulb to the ligament of Treitz. The internal tubular implant one of more expandable rings or stents to anchor the device inside of the duodenal bulb.

FIGS. 32-33 show tubular implants or sleeves including one or more magnets for attachment.

FIGS. 34-39 show various embodiments of an internal tubular implant sleeve.

FIG. 40 shows a stent which may be used as an anchoring device for internal tubular implant. The stent may incorporate magnets that attract or repel portions of the external band. The stent device may also anchor the sleeve of the internal tubular implant by mechanical means.

FIGS. 41A-48 show various embodiments of a stent that may be used as an anchoring device for an internal tubular implant.

FIG. 49 shows a delivery device for an internal tubular implant that is designed to go over the outside of an endoscope. The delivery device is loaded over the outside of an endoscope.

FIGS. 50-51 show various embodiments of a delivery device for an internal tubular implant.

FIG. 52 shows a cross-sectional view of a portion of the digestive tract in the body. An external anchoring device is implanted around the esophagus at the gastro-esophageal junction. An internal tubular implant is implanted at the gastro-esophageal junction. The internal tubular implant can serve the function of an anti-reflux valve or a restrictive stoma.

FIG. 53 is a sectional view of a portion of the digestive tract in the body in the region of the junction of the stomach and the small intestine.

FIG. 54 is a sectional view of a portion of the digestive tract in the body showing an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum and/or the jejunum, beyond the ligament of Treitz.

FIG. 55 shows the internal implant depicted in FIG. 54 in detail.

FIG. 56 shows the external band depicted in FIG. 54 in detail.

FIG. 57 is a sectional view of a portion of the digestive tract in the body showing an alternate embodiment of an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum beyond the ligament of Treitz.

FIG. 58 shows the external band depicted in FIG. 57 in detail.

FIG. 59 is a sectional view of a portion of the digestive tract in the body showing an alternate embodiment of an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum beyond the ligament of Treitz.

FIG. 60 shows the internal implant depicted in FIG. 59 in detail.

FIG. 61 is a sectional view of a portion of the digestive tract in the body showing an alternate embodiment of an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum beyond the ligament of Treitz.

FIG. 62 shows the external band depicted in FIG. 61 in detail.

FIG. 63 is a sectional view of a portion of the digestive tract in the body showing an alternate combination of an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum beyond the ligament of Treitz.

FIG. 64 is a sectional view of a portion of the digestive tract in the body showing an alternate embodiment of an external band implanted around the outside diameter of the duodenal bulb and a tubular implant (sleeve) implanted on the inside surface of the duodenal bulb and anchored mechanically through the duodenal bulb tissue to the external band. The tubular implant extends into the duodenum beyond the ligament of Treitz.

FIG. 65 shows the external band depicted in FIG. 64 in detail.

FIGS. 66A, 66B, 67A, 67B and 68 show steps in the procedure a physician would perform in order to place the external band laparoscopically around the duodenal bulb followed by the precise placement of the internal implant with the sleeve attached under endoscopic and fluoroscopic guidance so that the internal implant interlocks with the external band.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

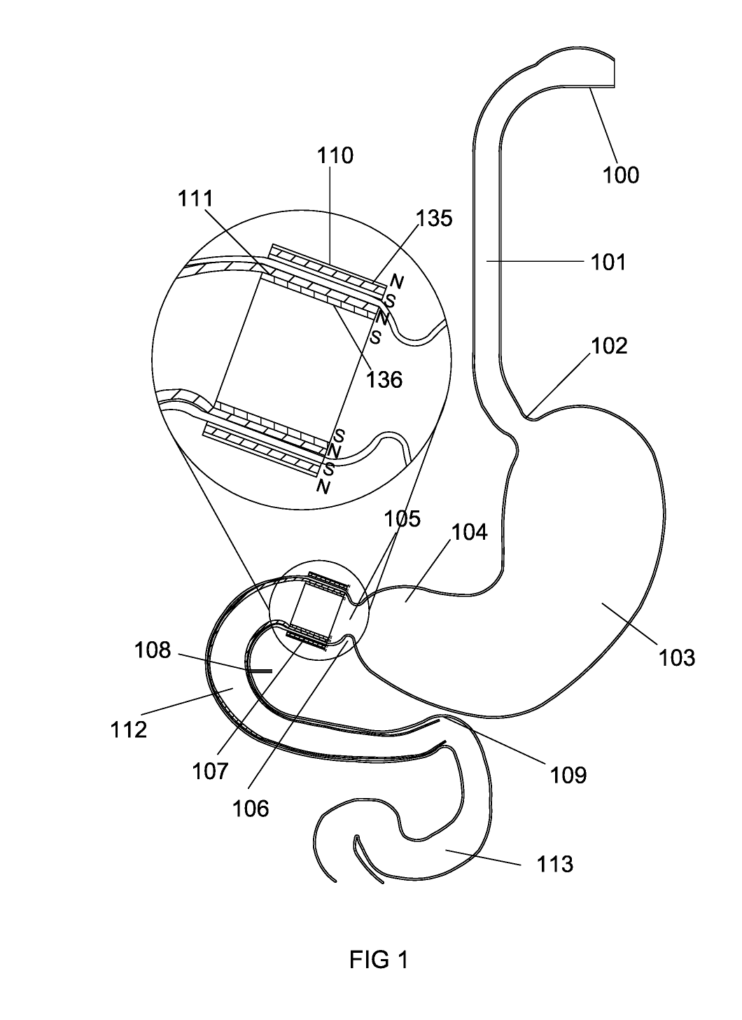

FIG. 1 is a schematic, sectional view of a portion of a human digestive tract. As a person ingests food, the food enters the mouth 100, is chewed, and then proceeds down the esophagus 101 to the lower esophageal sphincter at the gastro-esophageal junction 102 and into the stomach 103. The food mixes with enzymes in the mouth 100 and in the stomach 103. The stomach 103 converts the food to a semi-fluid substance called chyme. The chyme enters the pyloric antrum 104 and exits the stomach 103 through the pylorus 106 and pyloric orifice 105. The small intestine is about 21 feet long in adults and is comprised of three sections: the duodenum 112, the jejunum 113 and the ileum (not shown). The duodenum 112 is the first portion of the small intestine and is typically 10-12 inches long. The duodenum 112 is comprised of four sections: the superior, descending, horizontal and ascending. The duodenum 112 ends at the ligament of Treitz 109. The papilla of Vater 108 is the duct that delivers bile and pancreatic enzymes to the duodenum 112. The duodenal bulb 107 is the portion of the duodenum which is closest to the stomach 103.

As shown, an external band 110 is implanted around the duodenal bulb 107 and an internal tubular implant 111 is attached to the external band and extended into the duodenum 112 (e.g., to the ligament of Treitz 109). Magnets 135 on the external band 110 and magnets 136 on the internal tubular implant 111 magnetically interact with (e.g., attraction, repulsion, or levitation) each other and secure the internal tubular implant to the 111 to the external band implant 110 in a removable or reversible configuration. The external band 110 and the internal implant 111 anchor by coupling with each other using any of a variety of techniques including, for example, anchoring means that are based on mechanical interference, elasticity, spring force, shape memory transformation, magnetic attraction, repulsion and/or levitation.

According to some embodiments, the internal implant 111 is configured such that it exerts little or no radial force against an internal surface of the gastrointestinal tract. Likewise, according to various embodiments, the external band is sized and shaped such that, in its final implant configuration, it exerts little or no radial force against an external surface of the gastrointestinal tract. For example, in certain embodiments, the external band has an implanted inner diameter generally equal to an outer diameter of the desired mounting location of the gastrointestinal tract. More specifically, in some such embodiments, the implanted inner diameter of the external band is within ten percent of the corresponding outer diameter of the implant location of the gastrointestinal tract. In other such embodiments, the implanted inner diameter of the external band is within five percent of the corresponding outer diameter of the implant location of the gastrointestinal tract. In other such embodiments, the implanted inner diameter of the external band is within two percent of the corresponding outer diameter of the implant location of the gastrointestinal tract.

According to various embodiments, as a second mode of anchoring, stabilizing, or preventing migration, the external band 110 is coupled to an anatomical feature (e.g., a ligament) external to the tissue of the esophagus, stomach, pylorus, or intestine. In some embodiments, the external band 110 is intertwined with or threaded between the anatomical feature and the tissue. According to one exemplary embodiment, the external band 110 is coupled to the hepatoduodenal ligament. This second mode of anchoring enables the use of external bands that do not rely on excessive compressive force to keep the implant in place, since excessive compressive forces can cause tissue necrosis and erosion.

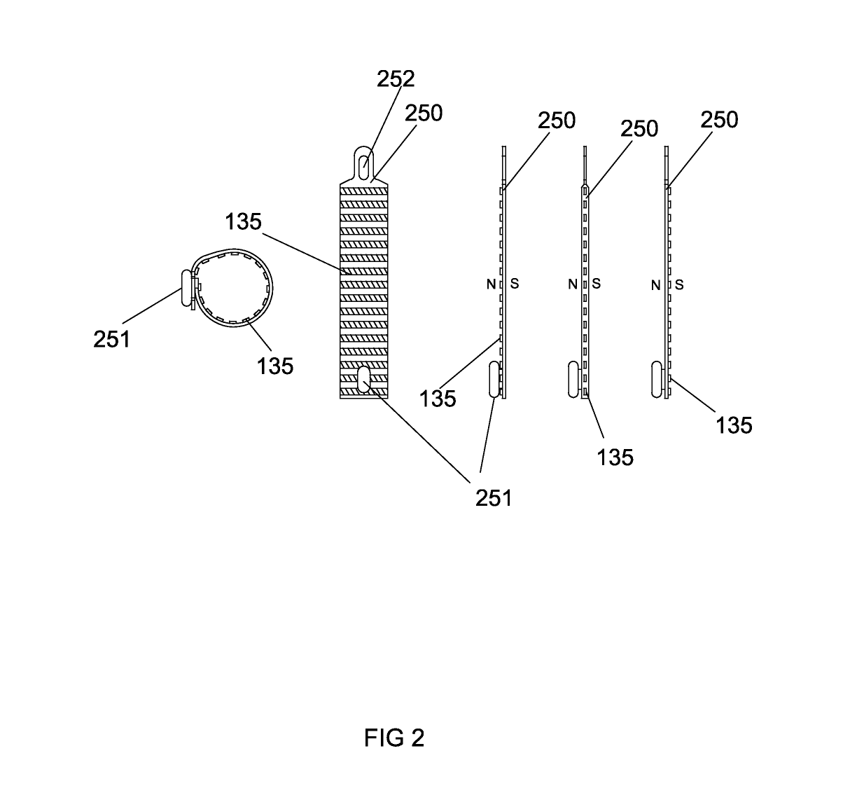

FIG. 2 shows an external band that may be used to provide for an external anchoring device to secure the internal tubular implant. The band 250, according to various embodiments, is made from one or more elastomers (e.g., silicone, polyurethane, and ePTFE), metals or fabrics (e.g., Dacron or a combination of polymers and textile materials). The band can be made flexible with varying degrees of longitudinal elasticity. According to various embodiments, the external band includes magnets 135 located on the inside surface, outside surface, or embedded in the middle of the band. Suitable materials for the magnets include, for example: neodymium-iron-boron [Nd—Fe—B], samarium-cobalt [Sm—Co], alnico, and hard ferrite [ceramic] or other suitable material. The magnets may be plated with gold or platinum or other material to make them radio-opaque or to improve corrosion resistance. The magnets may be encapsulated within a metal casing such as titanium or stainless steel to improve the corrosion resistance and the biocompatibility. As shown in FIG. 2, the magnets 135 are shaped as bars, which may have a length generally similar to the width of the band 250. According to various embodiments, the band is placed around the intended implant location (e.g., the duodenal bulb, esophagus, or stomach). The band, according to various embodiments, is wrapped around the implant location like a belt. As shown, for example, in FIG. 2, one end of the band 250 has a loop 252 and the other end has a button 251. The band is enclosed around the duodenal bulb or other implant location, the loop 252 is snapped over the button 251 to secure the band closed. The magnetic poles of the magnet are aligned such that the all the north poles are aligned to the inside or outside of the band. The magnets on the inside tubular implant are assembled to the device, so the poles are the opposite magnetic polarity, and the outside band and the inside tubular implanted will attract to each other when one device is sleeved inside the other.

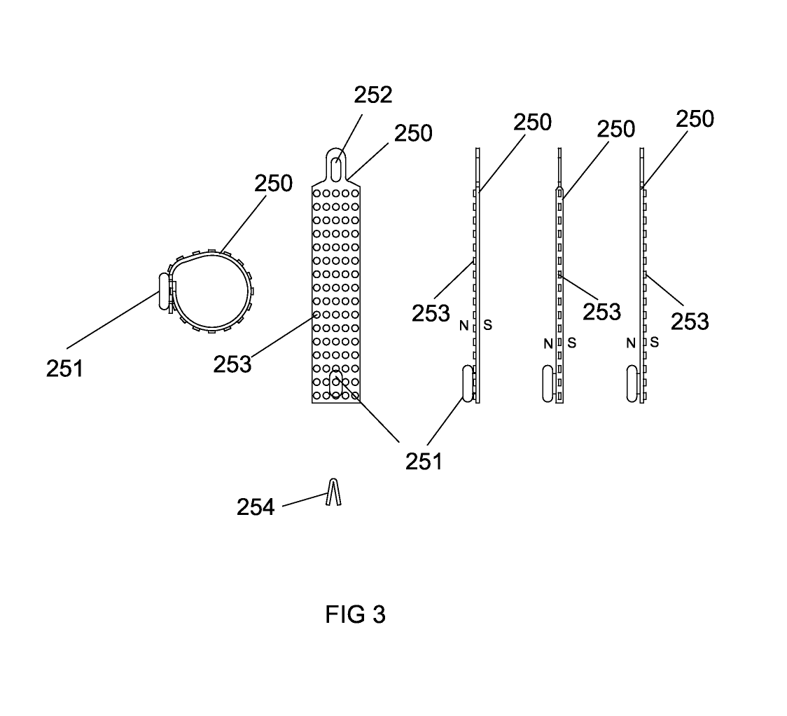

FIG. 3 shows an alternative embodiment of an external band implant 110 in which the magnet 253 shape is changed to be a round disk. The round disk will provide for a band which is easier to longitudinally fold as in 254. This fold will make it easier to insert the band in through a trocar when minimally invasive surgery techniques are used to implant the band.

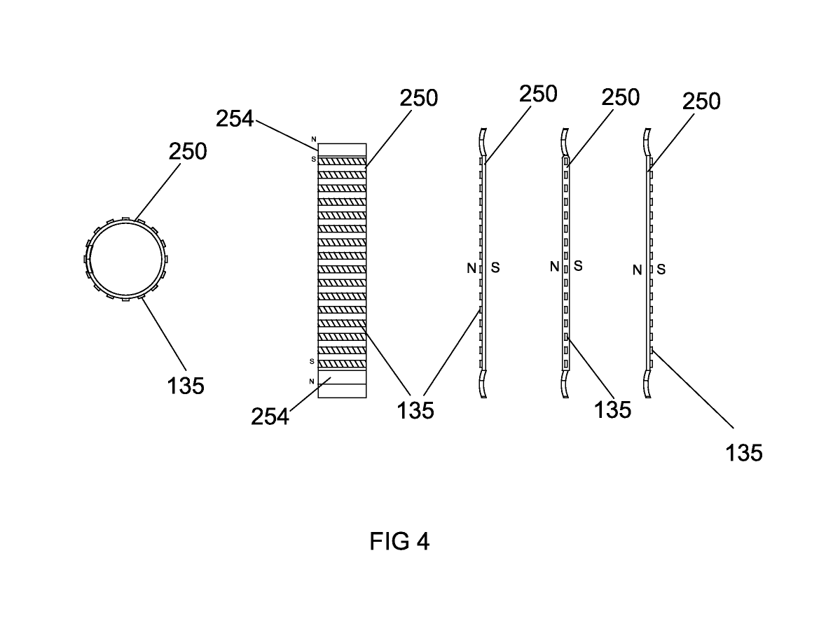

FIG. 4 shows an alternative embodiment of an external band implant 110 with the magnet 135 shaped as in FIG. 2. The band has a magnetic clasp for securing the band 254.

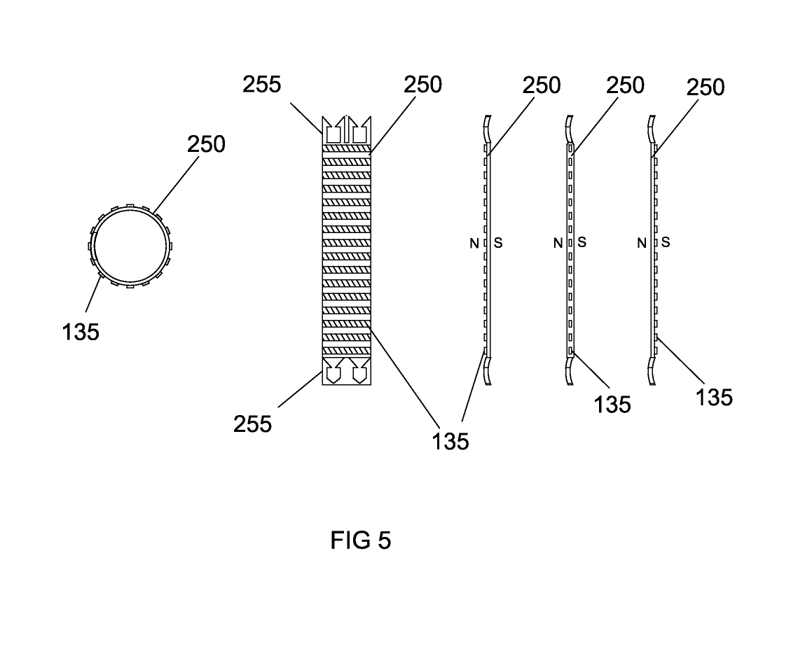

FIG. 5 shows an alternative embodiment of the external band implant 110 with the magnet 135 shaped as in FIG. 2. As shown in FIG. 5, the band has a mechanical clasp for securing the band 255.

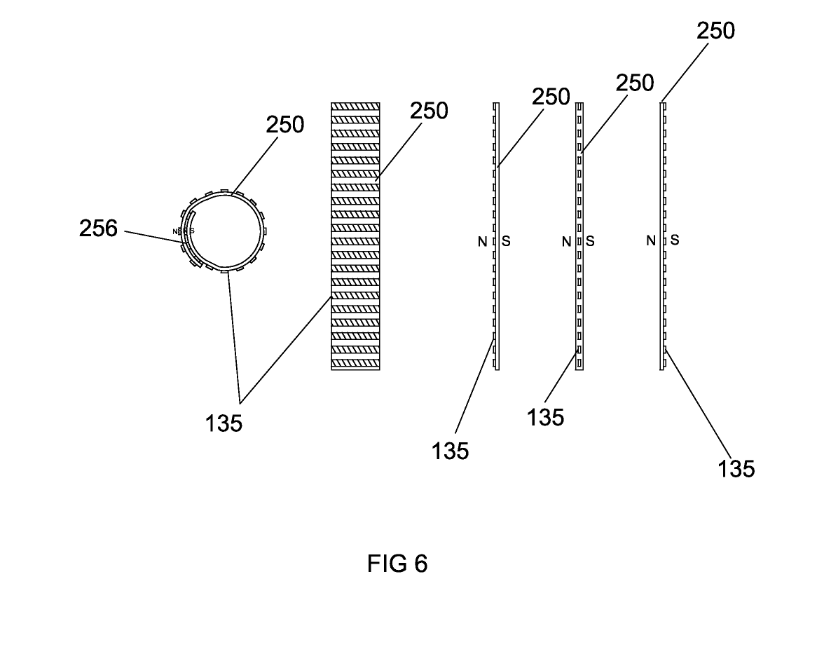

FIG. 6 shows another alternative embodiment of an external band implant 110. The two ends of the band are secured by overlapping the band and the magnets on the inner layer of the band and the outer layer of the band are attracted to each other to hold and secure the band. The band is adjustable in size by changing the length of the overlap 256.

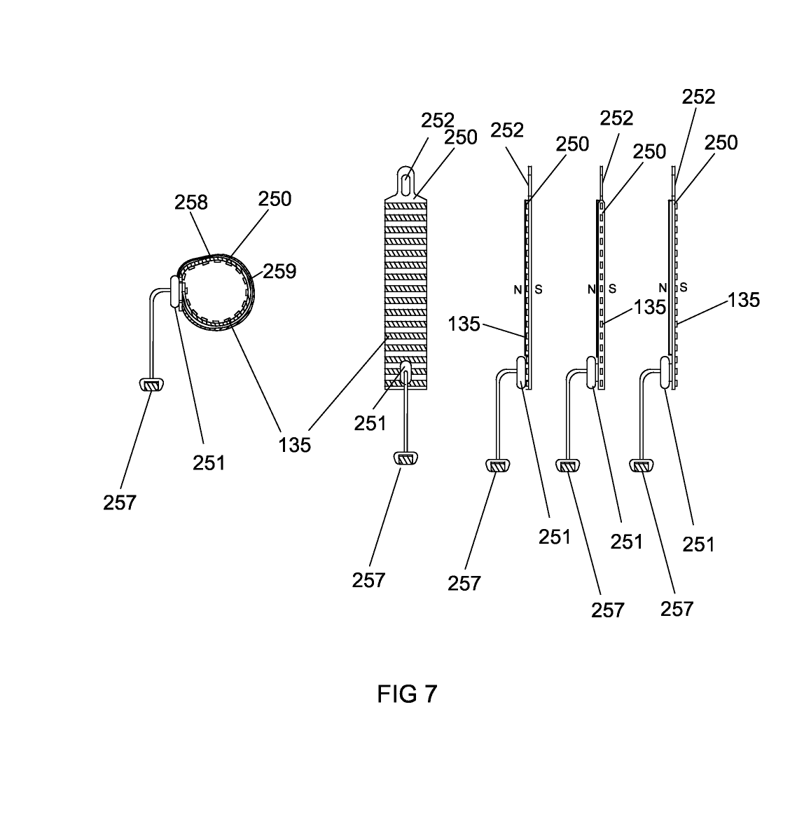

FIG. 7 shows another alternative embodiment of an external band implant 110. The band is secured with the same means as in FIG. 2. The band has been modified to have two layers. The inner layer 250 is similar to FIG. 2, but there is now also an outer band 258. The two bands 250 and 258 form an annular space 259 in between the two bands. This annular space can be filled with air, saline or other suitable material to cause the band to inflate balloon like with the main expansion inward to reduce the inside diameter of the band. The fluid in the device can be adjusted by inserting a needle through the septum 257. Additional fluid may be added or removed through the septum to change the sizing of the band.

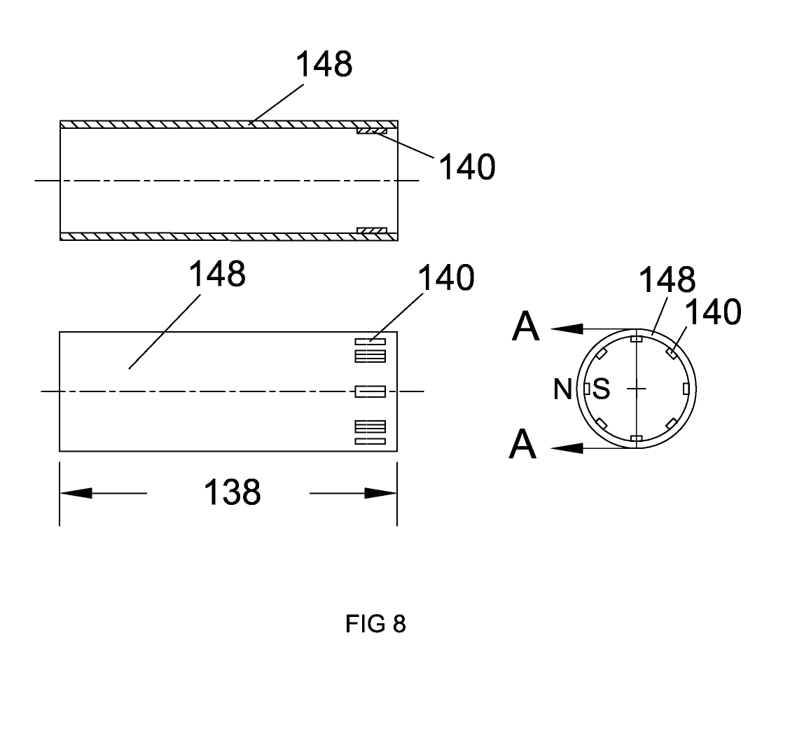

FIG. 8 shows an internal tubular implant that can be used to bypass the stomach 103, duodenum 112 or other intestinal lumen. The tubular implant is made of a thin wall tube 148 and a series of magnets 140 attached to the inside of the thin wall tube. The tubular implants, according to various embodiments, are made from one or more of the following materials: silicone, polyether block amides (PEBAX), polyurethanes, silicone polyurethane copolymers, Nylon, polyethylene terphalate (PET), ePTFE, Kevlar, Spectra, Dyneena, polyvinyl chloride (PVC), polyethylene, polyester elastomers or other suitable materials. The thin wall tube length, according to various embodiments, may range from about 1 inch in length up to about 5 feet in length. The thickness of the thin walled tube will typically be in the range of from about 0.0001 inch thick up to about 0.10 inch thick. The diameter of the tubular implant will typically range from about 25 mm to about 35 mm, with a maximum range anticipated of from about 5 mm to about 70 mm in diameter.

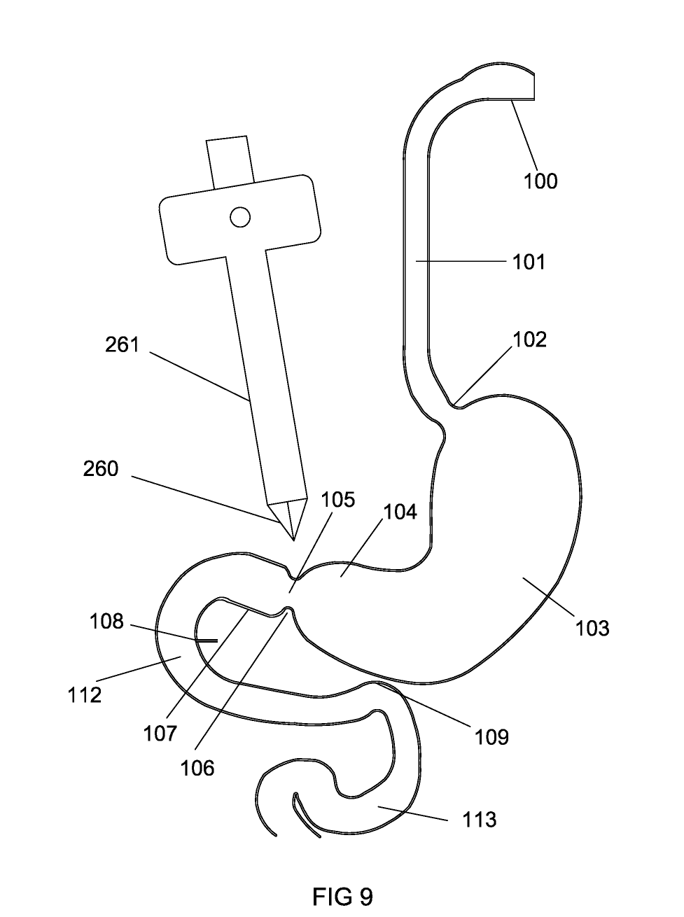

FIG. 9 shows a cross-sectional view of a portion of the digestive tract in the body with a trocar 260 and cannula 261 inserted to access the implant location of the duodenal bulb using laparoscopic techniques. An alternative access route is to use natural orifice surgery via the esophagus, stomach or vagina.

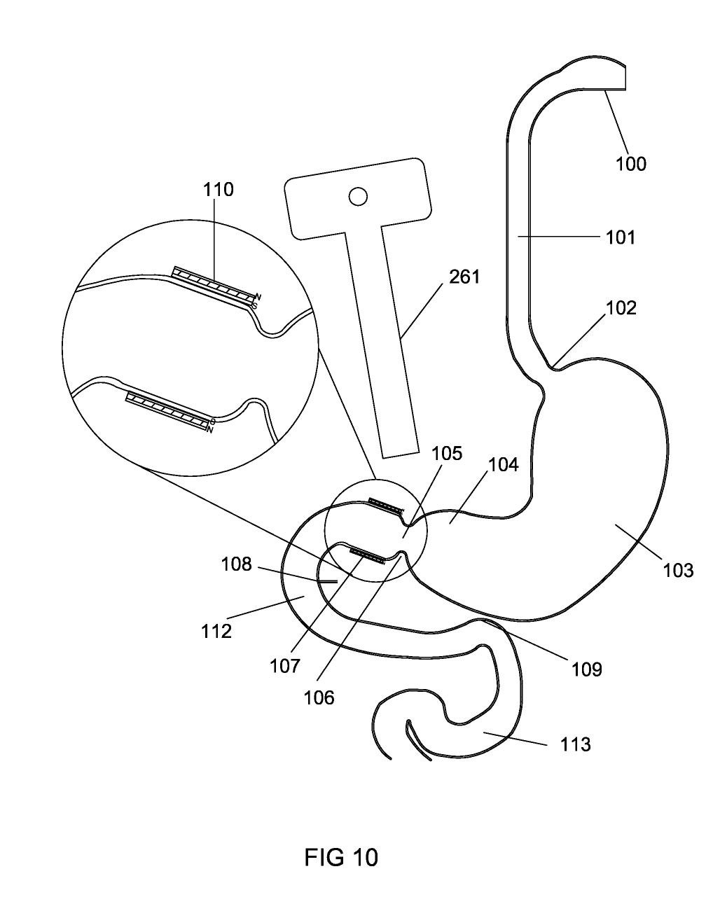

FIG. 10 shows a cross-sectional view of a portion of the digestive tract in the body with the trocar removed and cannula 261 inserted to access the implant location of the duodenal bulb using laparoscopic techniques. As shown, the external band 110 has been implanted around the duodenal bulb.

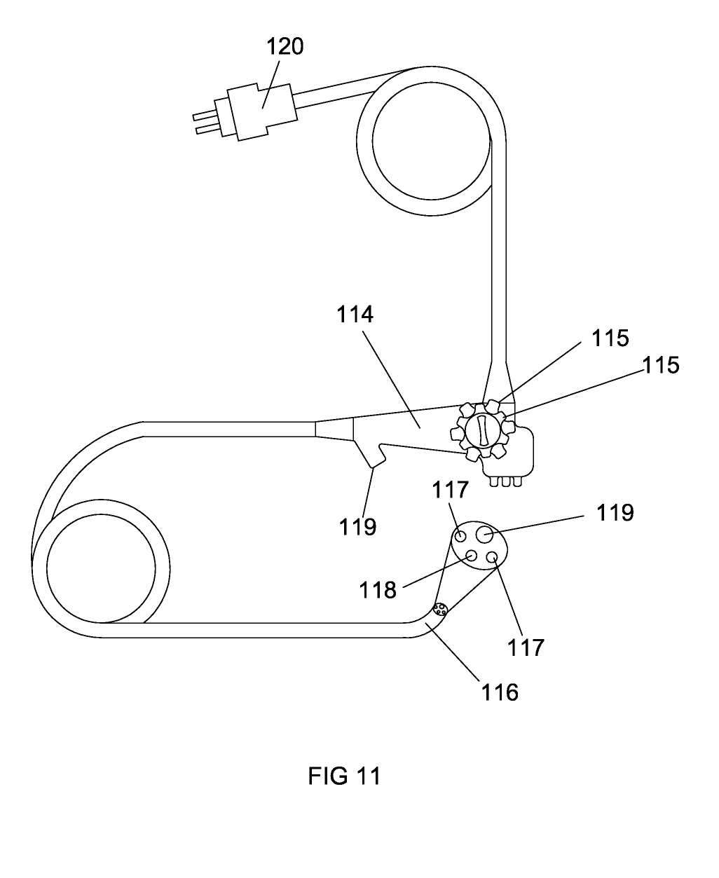

FIG. 11 shows an endoscope 114. Endoscopes 114 are used for diagnostic and therapeutic procedures in the gastrointestinal (GI) tract. The typical endoscope 114 is steerable by turning two rotary dials 115 to cause deflection of the working end 116 of the endoscope. The working end of the endoscope 116 or distal end, typically contains two fiber bundles for lighting 117, a fiber bundle for imaging 118 (viewing) and a working channel 119. The working channel 119 can also be accessed on the proximal end of the endoscope. The light fiber bundles and the image fiber bundles are plugged into a console at the plug in connector 120. The typical endoscope has a working channel in the 2.6 mm to 3.2 mm diameter range. The outside diameter of the endoscopes are typically in the 8 mm to 12 mm diameter range, depending on whether the endoscope is for diagnostic or therapeutic purposes.

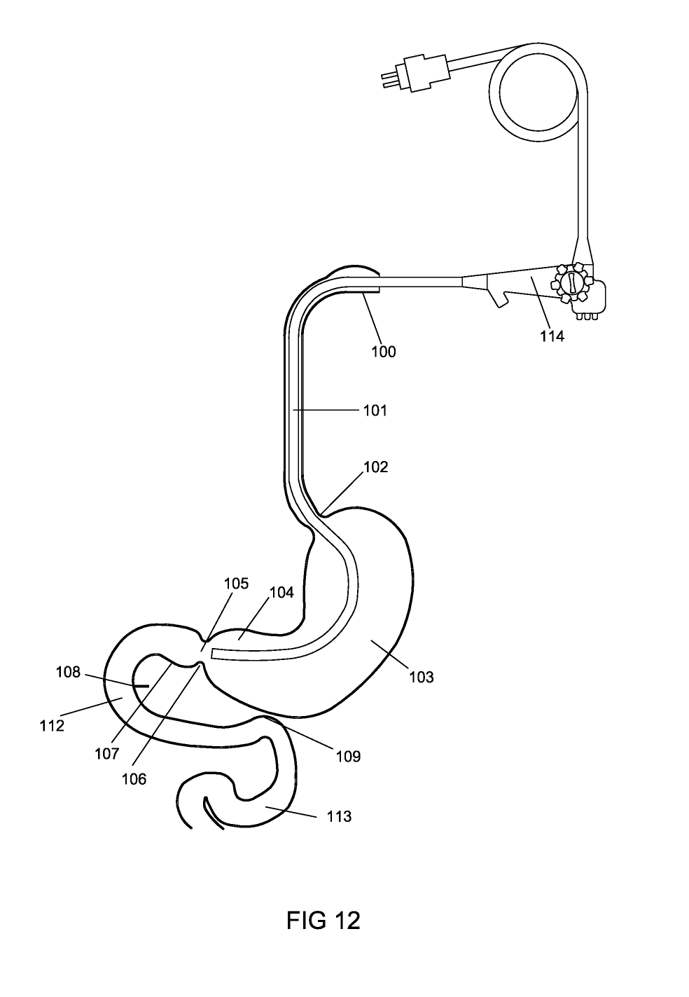

FIG. 12 is a cross-sectional view of a portion of the digestive tract in a human body. An endoscope 114 has been inserted through: the mouth 100, esophagus 101, stomach 103 and pyloric antrum to allow visualization of the pylorus 106.

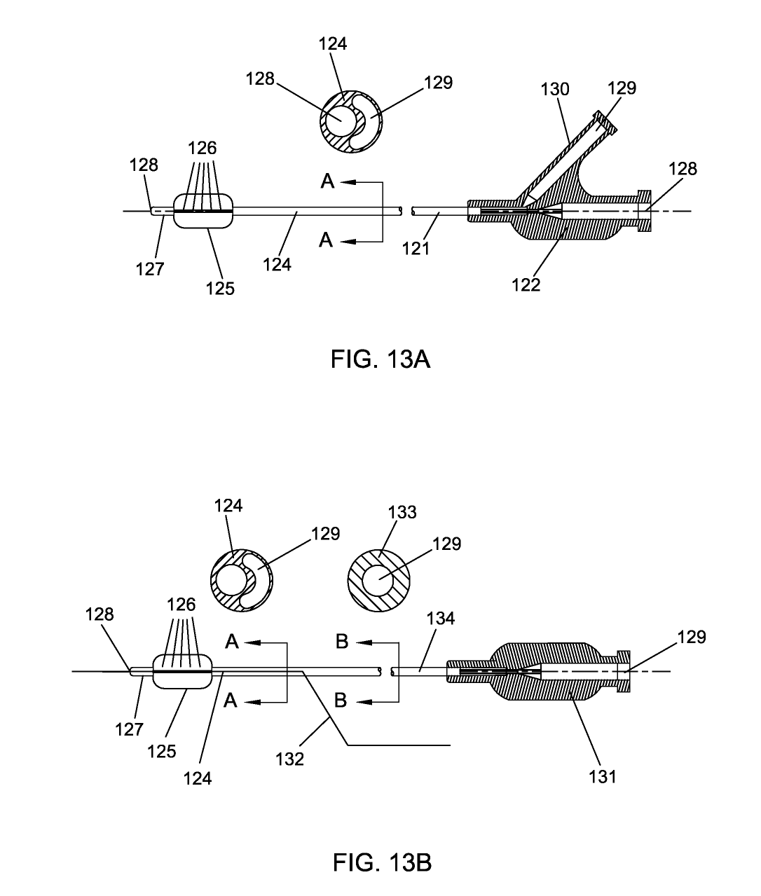

FIG. 13A shows an over the wire sizing balloon 121 that is used to measure the diameter of the pylorus 106, duodenal bulb 107, esophagus 102, pyloric antrum 104 or other lumen in the GI tract. The sizing balloon is composed of the following elements: proximal hub 122, catheter shaft 124, distal balloon component 125, radiopaque marker bands 126, distal tip 127, guide wire lumen 128, inflation lumen 129. Distal balloon component 125 can be made from silicone, silicone polyurethane copolymers, latex, nylon 12, PET (Polyethylene terphalate) Pebax (polyether block amide), polyurethane, polyethylene, polyester elastomer or other suitable polymer. The distal balloon component 125 can be molded into a cylindrical shape, into a dog bone or a conical shape. The distal balloon component 125 can be made compliant or non-compliant. The distal balloon component 125 can be bonded to the catheter shaft 124 with glue, heat bonding, solvent bonding, laser welding or suitable means. The catheter shaft can be made from silicone, silicone polyurethane copolymers, latex, nylon 12, PET (Polyethylene terphalate) Pebax (polyether block amide), polyurethane, polyethylene, polyester elastomer or other suitable polymer.

Section A-A in FIG. 13A shows a sectional view of the catheter shaft 124. The catheter shaft 124 is shown as a dual lumen extrusion with a guide wire lumen 128 and an inflation lumen 129. The catheter shaft 124 can also be formed from two coaxial single lumen round tubes in place of the dual lumen tubing. The balloon is inflated by attaching a syringe (not shown) to luer fitting side port 130. The sizing balloon accommodates a guidewire through the guidewire lumen from the distal tip 127 through the proximal hub 122. The sizing balloon can be filled with a radiopaque dye to allow visualization and measurement of the size of the anatomy with a fluoroscope. The sizing balloon 121 has two or more radiopaque marker bands 126 located on the catheter shaft to allow visualization of the catheter shaft and balloon position. The marker bands 126 also serve as a fixed known distance reference point that can be measured to provide a means to calibrate and determine the balloon diameter with the use of the fluoroscope. The marker bands can be made from tantalum, gold, platinum, platinum iridium alloys or other suitable material.

FIG. 13B shows a rapid exchange sizing balloon 134 that is used to measure the diameter of the pylorus 106, duodenal bulb 107, esophagus 102, pyloric antrum 104 or other lumen in the GI tract. The sizing balloon is composed of the following elements: proximal luer 131, catheter shaft 124, distal balloon component 125, radiopaque marker bands 126, distal tip 127, guide wire lumen 128, inflation lumen 129. The materials of construction will be similar to that of FIG. 4A. The guidewire lumen 128 does not travel the full length of the catheter, it starts at the distal tip 127 and exits out the side of the catheter at a distance shorter than the overall catheter length. Guidewire 132 is inserted into the balloon catheter to illustrate the guidewire path through the sizing balloon. The sizing balloon catheter shaft changes the section along its length from a single lumen at section B-B 133 to a dual lumen at section A-A at 124.

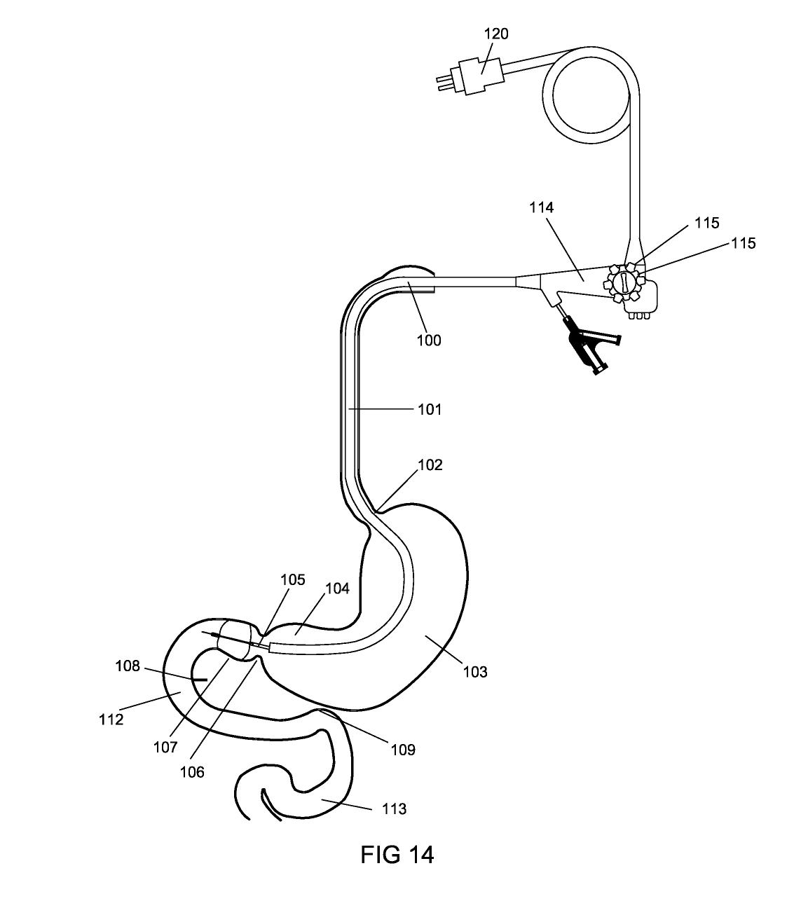

FIG. 14 shows an endoscope 114 inserted into the GI tract up to the pylorus 106. A sizing balloon 121 is inserted through the working channel 119 of the endoscope and into the area of the duodenal bulb 107. The sizing balloon 121 is inflated with a contrast agent. The diameter of the duodenal bulb 107 is measured with a fluoroscope.

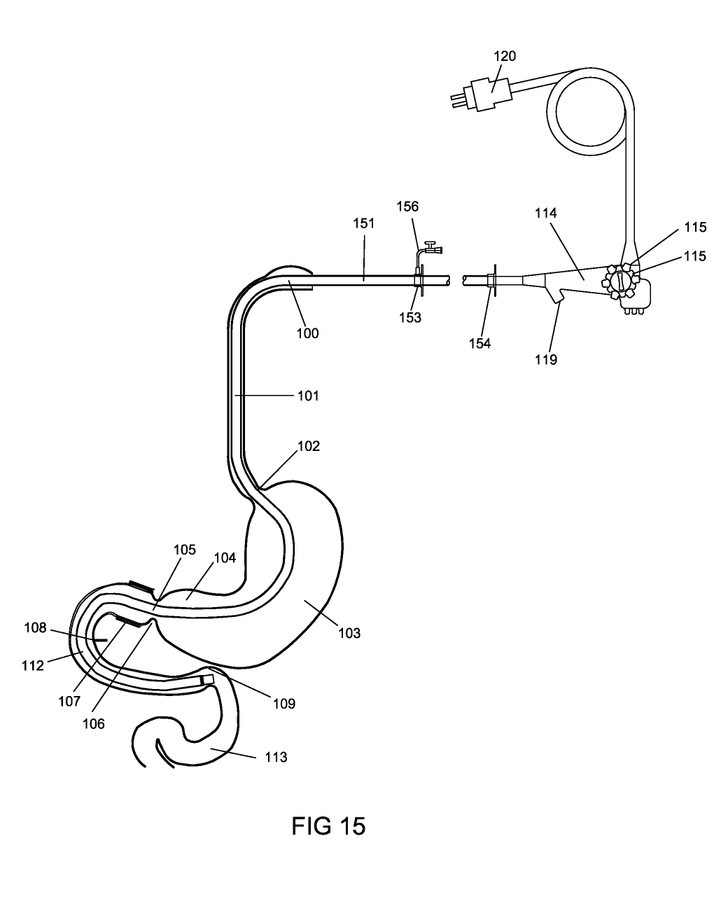

FIG. 15 shows a sectional view of a portion of the digestive tract in the body. An internal tubular implant is loaded onto the delivery catheter. The delivery catheter, according to various embodiments, is advanced into the duodenum 112 until the distal end of the delivery catheter is at the ligament of Treitz 109. While in many embodiments, the end of the delivery catheter is positioned at the ligament of Treitz 109, according to other embodiments, the end of the delivery catheter is located proximal or distal to the ligament of Treitz 109. For example, the distal end of the delivery catheter may be located in the jejunum 113. The internal tubular implant is deployed from the delivery catheter by pulling handle 153 towards 154.

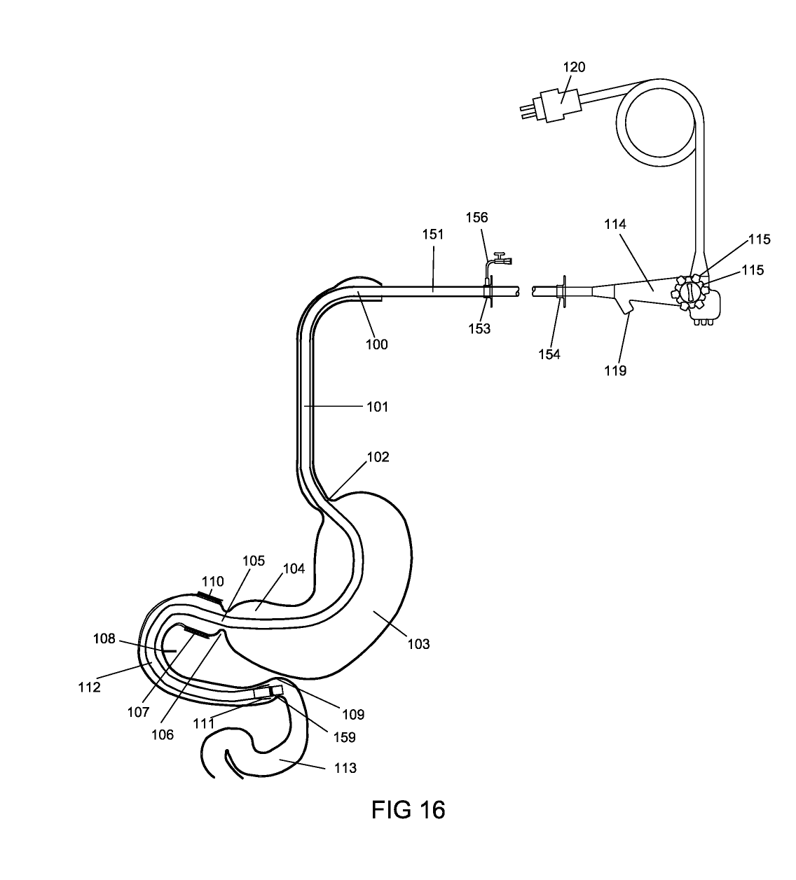

Next, as shown in FIG. 16, the outer sheath 151 on the delivery catheter is retracted a couple of inches to expose the tubular implant 111.

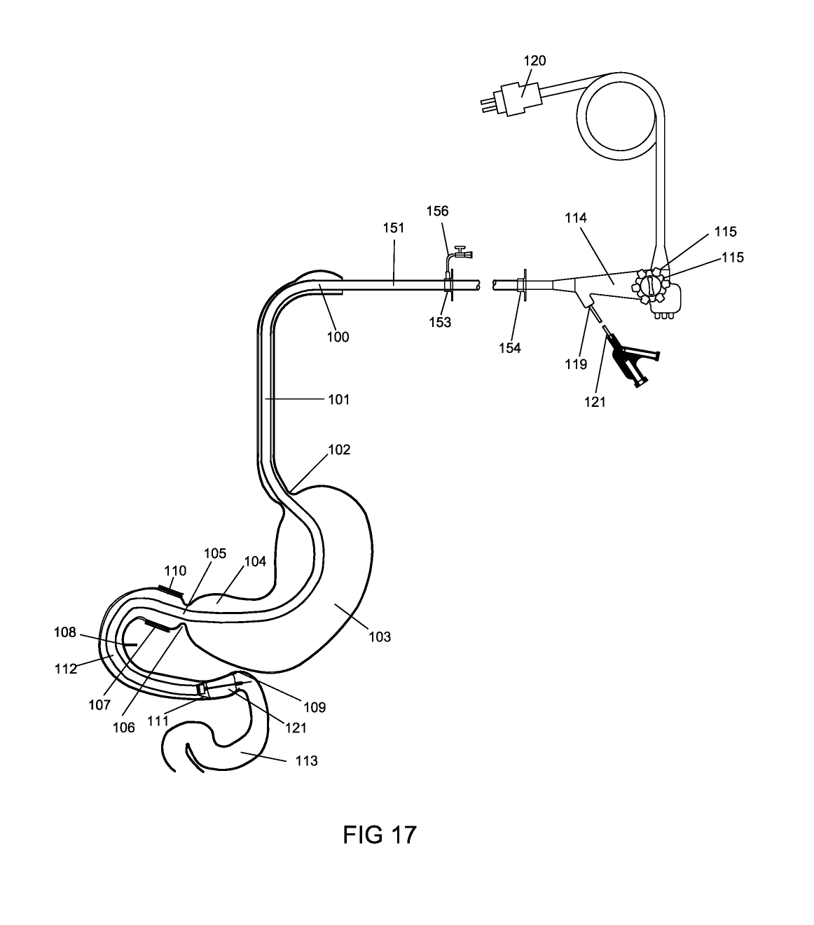

Next, as shown in FIG. 17, a sizing balloon 121 is inserted through the working channel 119 on endoscope 114. The sizing balloon 121 is advanced about one inch beyond the distal end of the endoscope 114 but still inside of the tubular implant 111. The sizing balloon 121 is then inflated with saline or contrast agent. The inflated sizing balloon 121 will hold the tubular implant 111 in place in the duodenum 112 or jejunum 113 (e.g., near the ligament of Treitz 109).

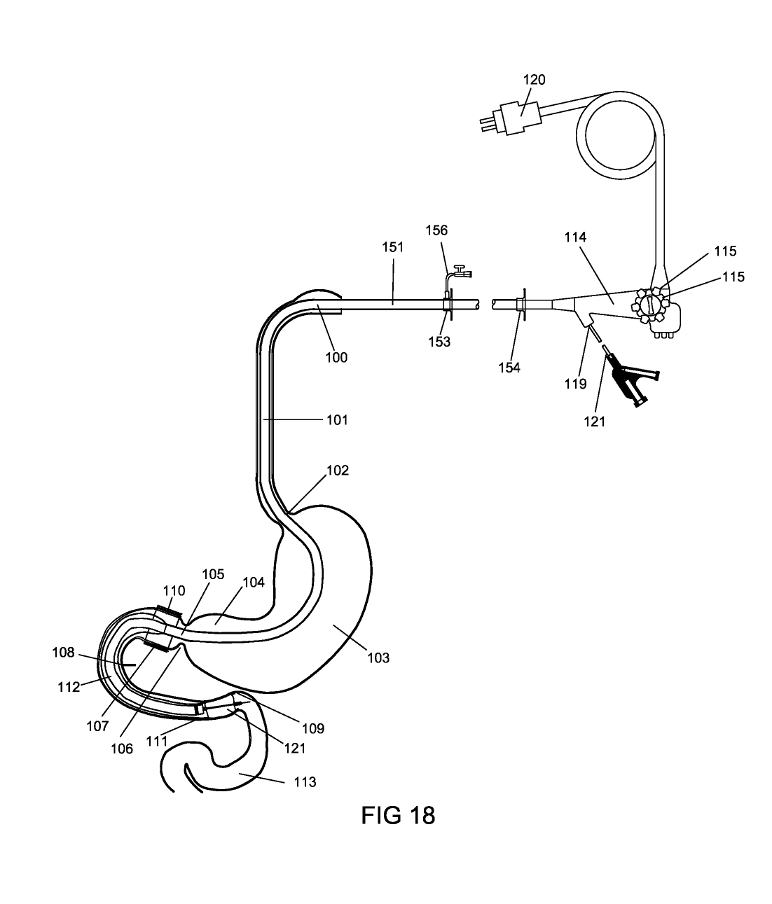

Then, as shown in FIG. 18, the outer sheath 151 is retracted further to expose all but a couple of centimeters of the tubular implant 111. The outer sheath 151 end is now near the pylorus 106.

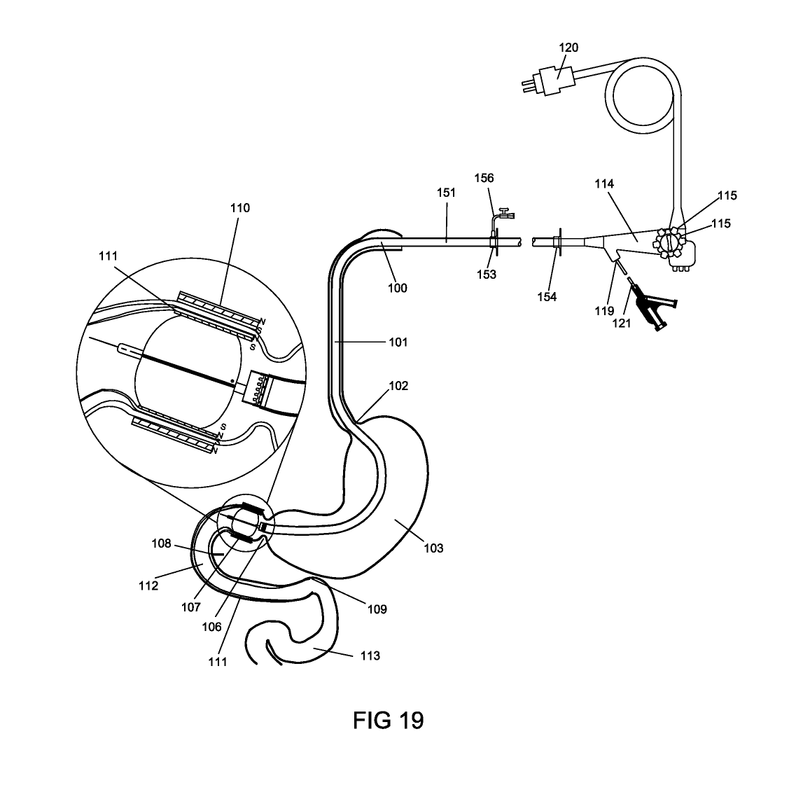

Then, as shown in FIG. 19, the distal end of the endoscope 114 has been pulled back to the pyloric orifice 105 and the sizing balloon 121 has been deflated and repositioned and reinflated to seat the proximal end of the internal tubular implant 111 to be in contact with the outer band 110. The magnets 140 on the tubular sleeve are now in contact with the magnets 140 on the docking element. The magnetic attraction between the magnets 140 secures the tubular implant 111 to the docking element 110.

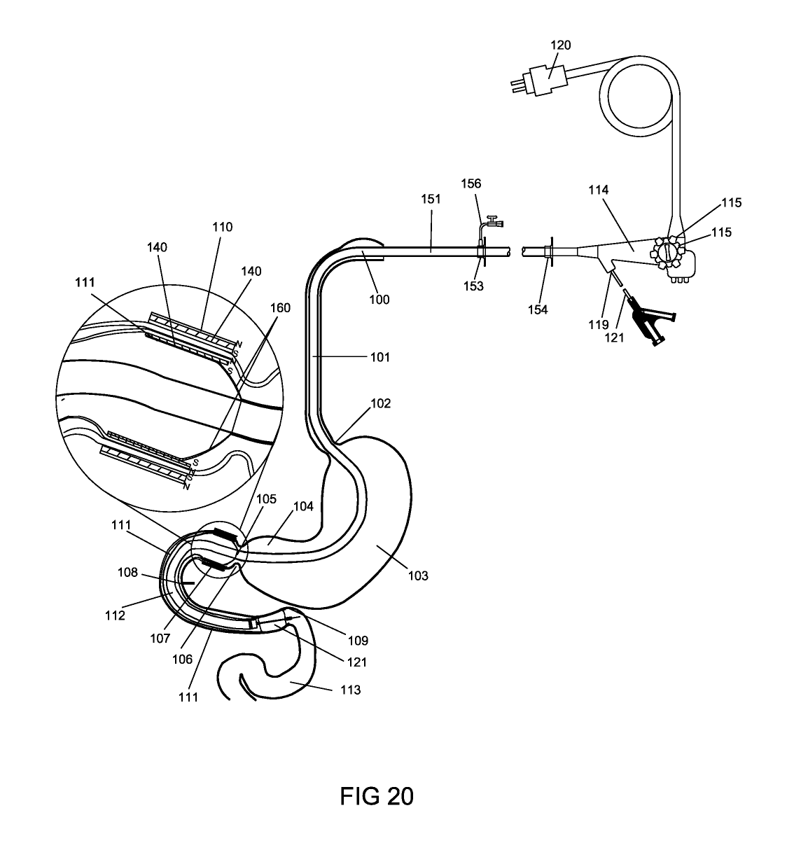

FIG. 20 is an alternative embodiment showing a means to seat the proximal end of the internal tubular implant 111 to the outer band 110. A Nitinol conical/tubular shaped forceps 160 are attached to the inner catheter near the proximal end of where the tubal implant is loaded on the delivery catheter. The Nitinol forceps 160 have an elastic memory in the open state. When the outer sheath 151 is fully retracted, the conical forceps open and, in turn, open the proximal end of the tubular implant 111 and seats the magnets 140 on the tubular implant 111 to the magnets 140 on the outer band 111.

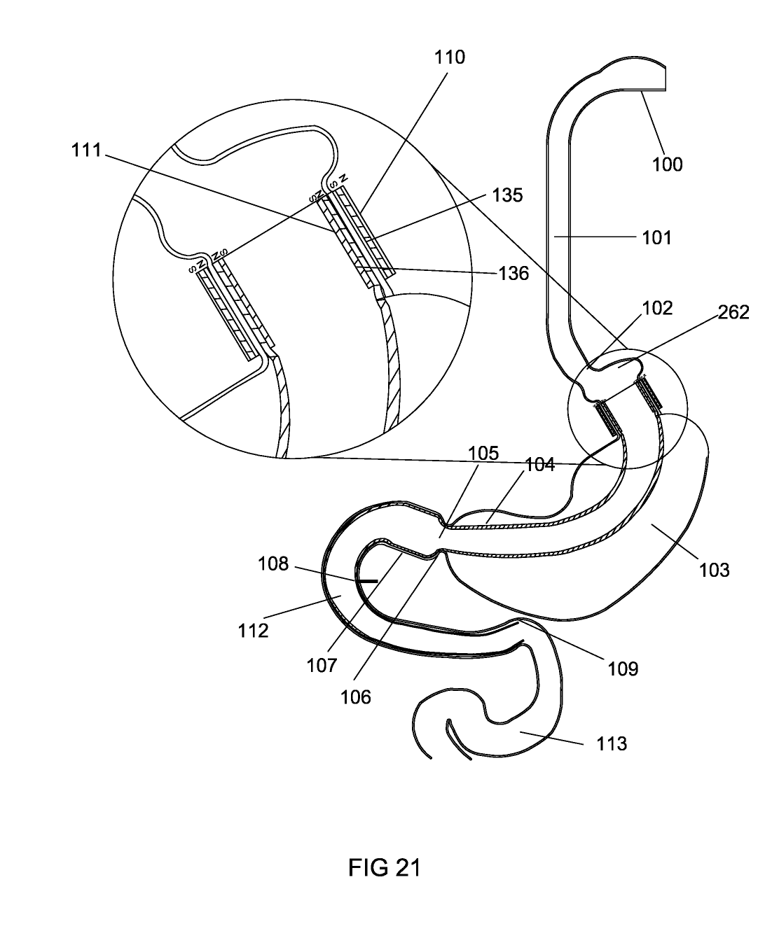

FIG. 21 shows a sectional view of a portion of the digestive tract in a human body. As shown, magnets 135 on the external band 110 and magnets 136 on the internal tubular implant 111 are magnetically attracted to each other and secure the internal tubular implant to the 111 to the external band implant 110. As shown, the external band 110 is secured to the tubular implant 111 at or near the gastro-esophageal junction. The external band 110 around the stomach creates a small pouch like area at the top of the stomach and causes a restrictive component to food flow into the digestive system. An external band 110 is implanted around the upper portion of the stomach and an internal tubular implant 111 is attached to the external band 110 and extended into the duodenum 112 or jejunum 113 (e.g., at or near the ligament of Treitz 109).

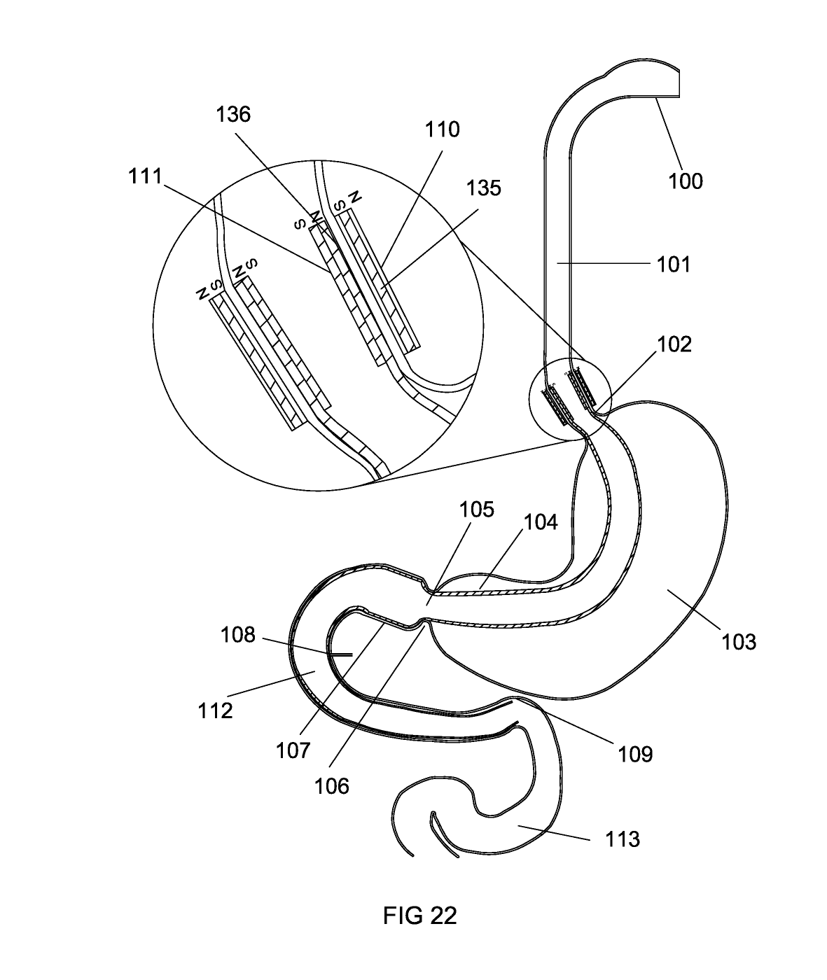

FIG. 22 shows a sectional view of a portion of the digestive tract in a human body. As shown, magnets 135 on the external band 110 and magnets 136 on the internal tubular implant 111 are magnetically attracted to each other and secure the internal tubular implant to the 111 to the external band implant 110. As shown, the external band 110 is secured to the tubular implant 111 at or near the gastro-esophageal junction. An external band 110 is implanted around the esophagus and an internal tubular implant 111 is attached to the external band 110 and extended into the duodenum 112 or the jejunum 113 (e.g., at or near the ligament of Treitz 109).

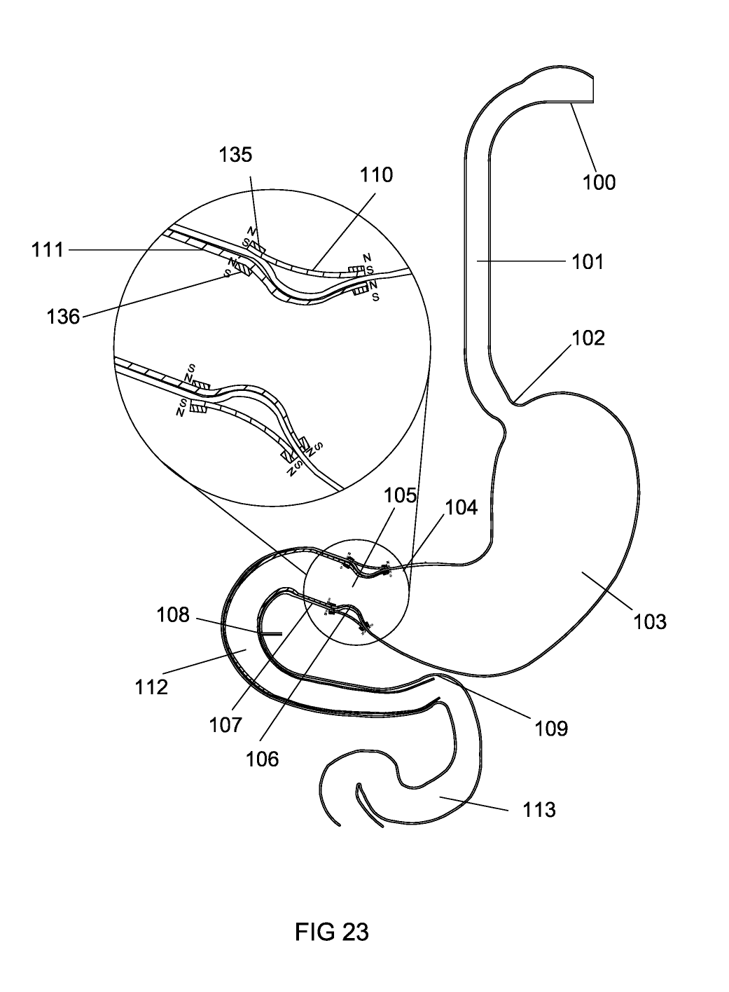

FIG. 23 shows a sectional view of a portion of the digestive tract in a human body. As shown, magnets 135 on the external band 110 and magnets 136 on the internal tubular implant 111 are magnetically attracted to each other and secure the internal tubular implant 111 to the external band implant 110. As shown, the external band 110 is secured to the tubular implant 111 at or near the gastrointestinal junction (e.g., across the pylorus) and the internal tubular implant 111 extends into the duodenum 112 or the jejunum 113 (e.g., to the ligament of Treitz 109).

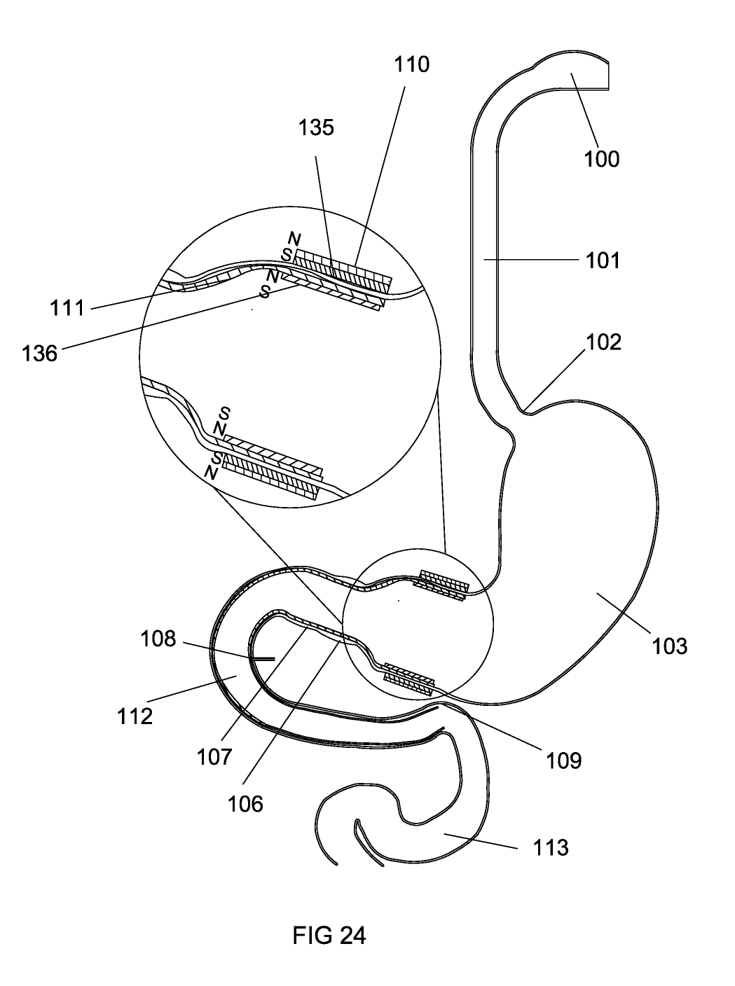

FIG. 24 shows a sectional view of a portion of the digestive tract in a human body. As shown, the external band 110 is implanted around the stomach antrum and an internal tubular implant 111 is attached to the external band 110 and is extended into the duodenum 112 or the jejunum 113 (e.g., to the ligament of Treitz 109). As shown, magnets 135 on the external band 110 and magnets 136 on the internal tubular implant 111 are magnetically attracted to each other and secure the internal tubular implant 111 to the external band implant 110.

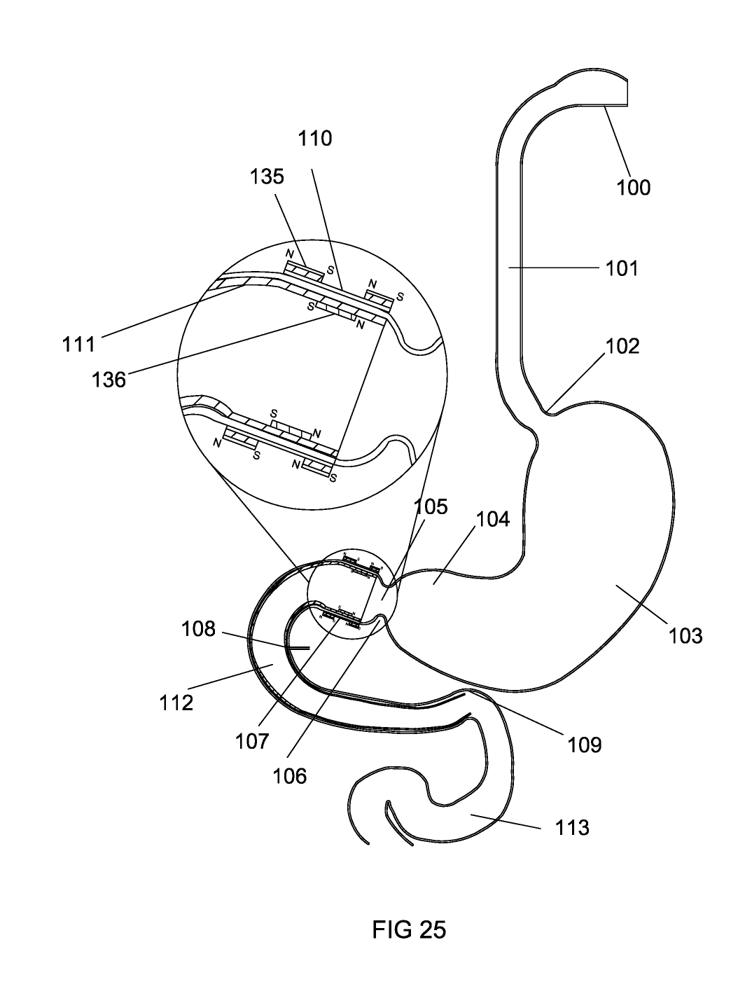

FIG. 25 shows a sectional view of a portion of the digestive tract in a human body. An external band 110 is implanted around the duodenal bulb 107 and an internal tubular implant 111 is attached to the external band 110 and extended into the duodenum 112 or the jejunum 113 (e.g., to the ligament of Treitz 109). Magnets 135 on the external band 110 and magnets 136 on the internal tubular implant 111 are magnetically repelled from each other and secure the internal tubular implant 111 to the external band implant 110.

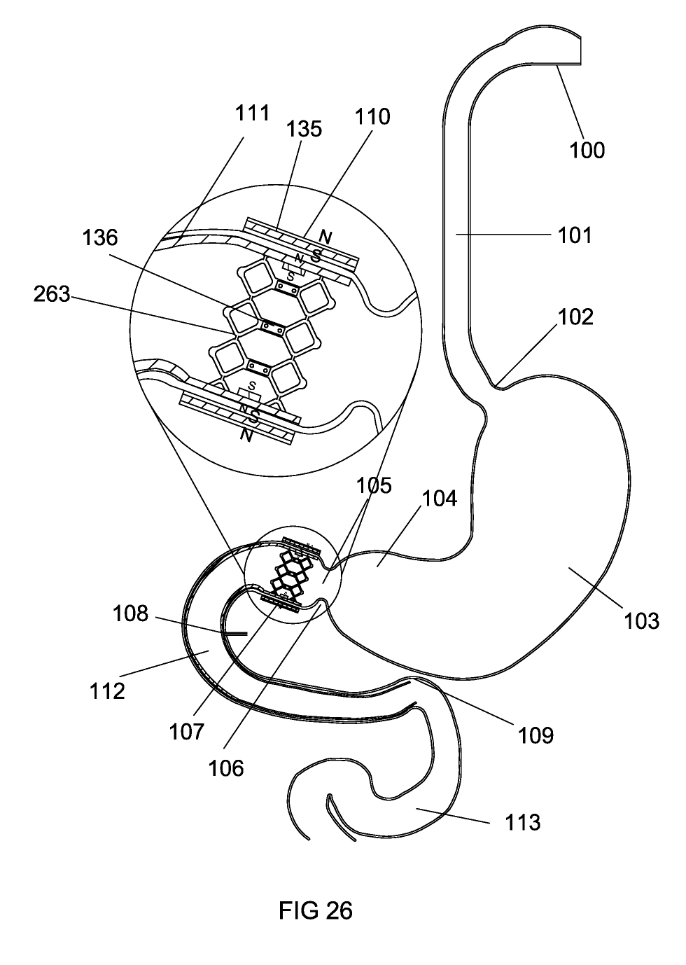

FIG. 26 is a sectional view of a portion of the digestive tract in a human body. An external band 110 is implanted around the duodenal bulb 107 and an internal tubular implant 111 is attached to the external band 110 and extended into the duodenum 112 or the jejunum 113 (e.g., to the ligament of Treitz 109). As shown, magnets 135 on the external band 110 and magnets 136 on the internal tubular implant 111 are magnetically attracted and secure the internal tubular implant 111 to the external band implant 110. In this embodiment, the internal tubular implant 111 includes and is coupled to a stent 263 (with magnets attached).

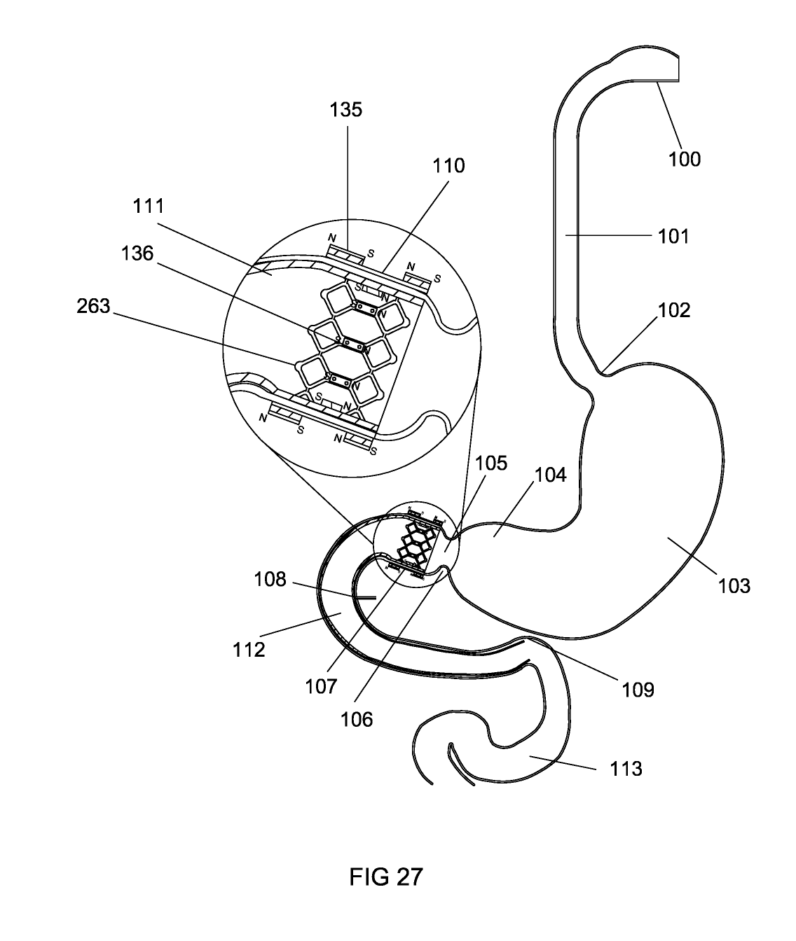

FIG. 27 is a sectional view of a portion of the digestive tract in a human body. An external band 110 is implanted around the duodenal bulb 107 and an internal tubular implant 111 is attached to the external band 110 and extended into the duodenum 112 or the jejunum 113 (e.g., to the ligament of Treitz 109). As shown, magnets 135 on the external band 110 and magnets 136 on the internal tubular implant 111 are magnetically repelled and secure the internal tubular implant 111 to the external band implant 110. Stent 263 (with magnets attached) is attached to internal tubular implant 111. In some embodiments, the stent 263 has an expanded outer diameter of generally equal to an inner diameter of the corresponding implant location of the gastrointestinal tract. According to some embodiments, the expanded outer diameter of the stent is within about ten percent, about five percent, or about two percent of the inner diameter of the corresponding implant location of the gastrointestinal tract.

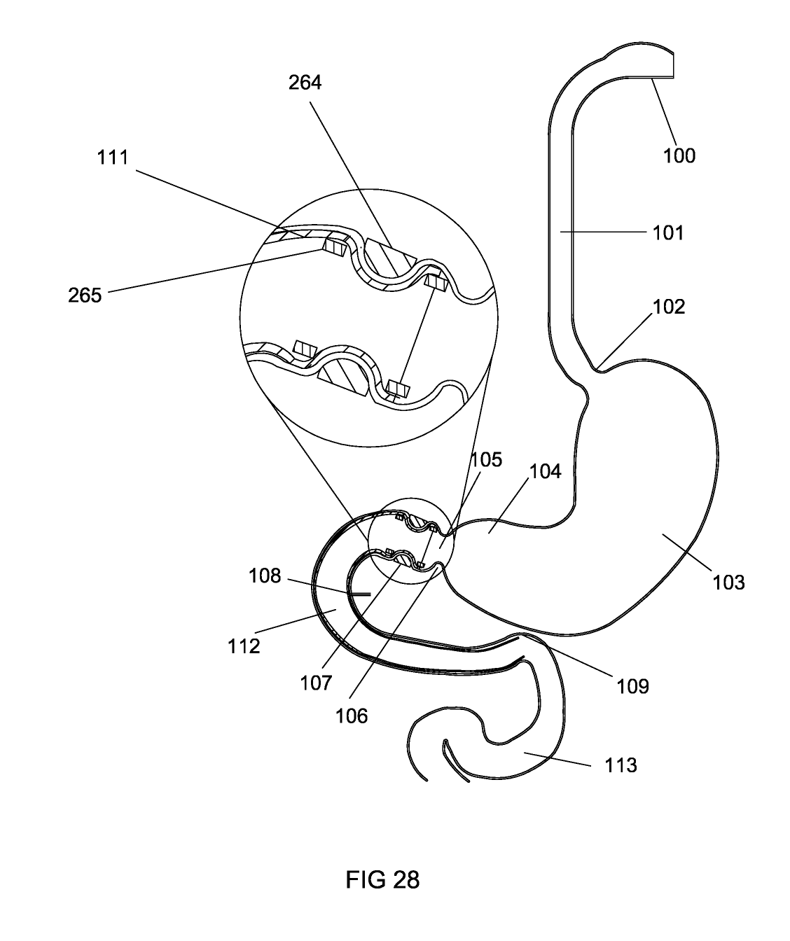

FIGS. 28-31 show various embodiments of an external band 264 and an internal implant 111 configured for removably or reversibly coupling with each other. As shown, in each of these embodiments, the internal implant includes a portion including a feature or a structure adapted to mechanically couple with a corresponding (e.g., mating) feature or structure of the external band 264. In each case, the coupling is accomplished through or across a gastrointestinal organ or tissue (e.g., across the duodenum, the pylorus, or the gastric antrum), but without penetrating such tissue. In some embodiments, the external band 264 and the internal implant 111 are not in direct mechanical contact, but instead engage or couple with each other with intervening gastrointestinal tissue.

FIG. 28 is a sectional view of a portion of the digestive tract in a human body. An external band 264 is implanted around the duodenal bulb 107 and an internal tubular implant 111 is attached to the external band 264 and extended into the duodenum 112 or the jejunum 113 (e.g., to the ligament of Treitz 109). As shown, diameter interference between at least a portion of the external band 264 and internal implant 110 limit longitudinal movement without exerting substantial radial force on the gastrointestinal tract. As shown, the tubular implant 111 includes an expandable ring or stent 265, which is attached to the tube portion of the tubular implant 111 and operates to secure the internal tubular implant 111 to the external band 264.

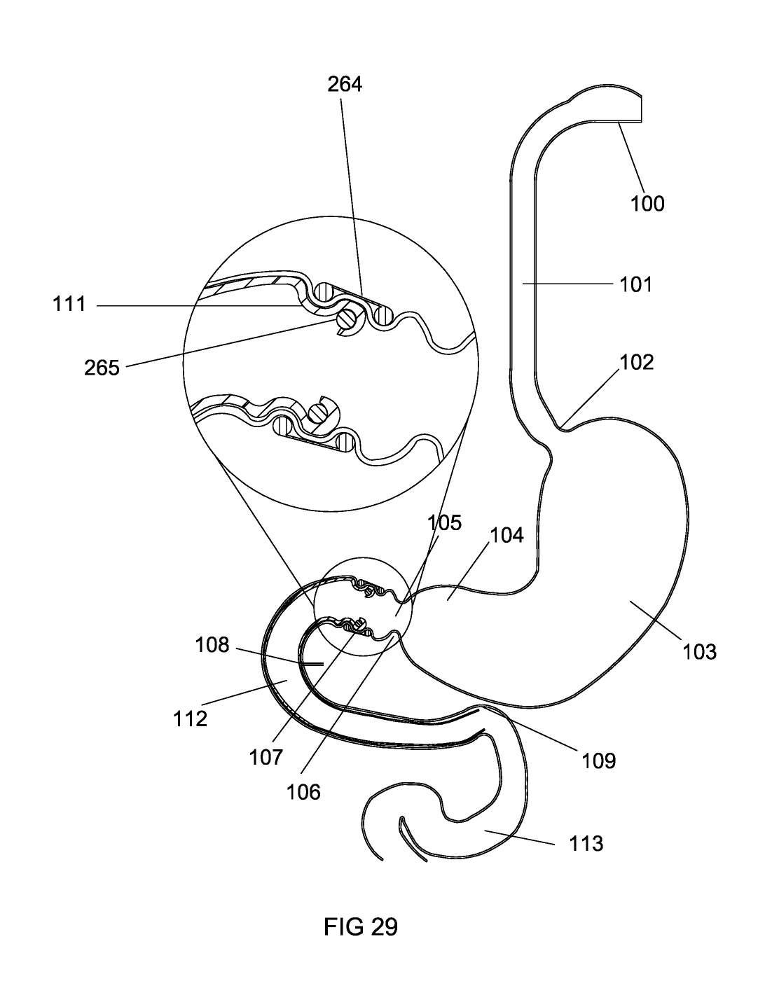

FIG. 29 is a sectional view of a portion of the digestive tract in a human body. An external band 264 is implanted around the duodenal bulb 107 and an internal tubular implant 111 is coupled to the external band and extended into the duodenum 112 or the jejunum 113 (e.g., to the ligament of Treitz 109). As shown, diameter interference with the external band 264 and internal implant 110 secure the implant 110 within the gastrointestinal system and limit longitudinal movement without exerting too much radial force. The tubular implant 111 includes an attached expandable ring or stent 265.

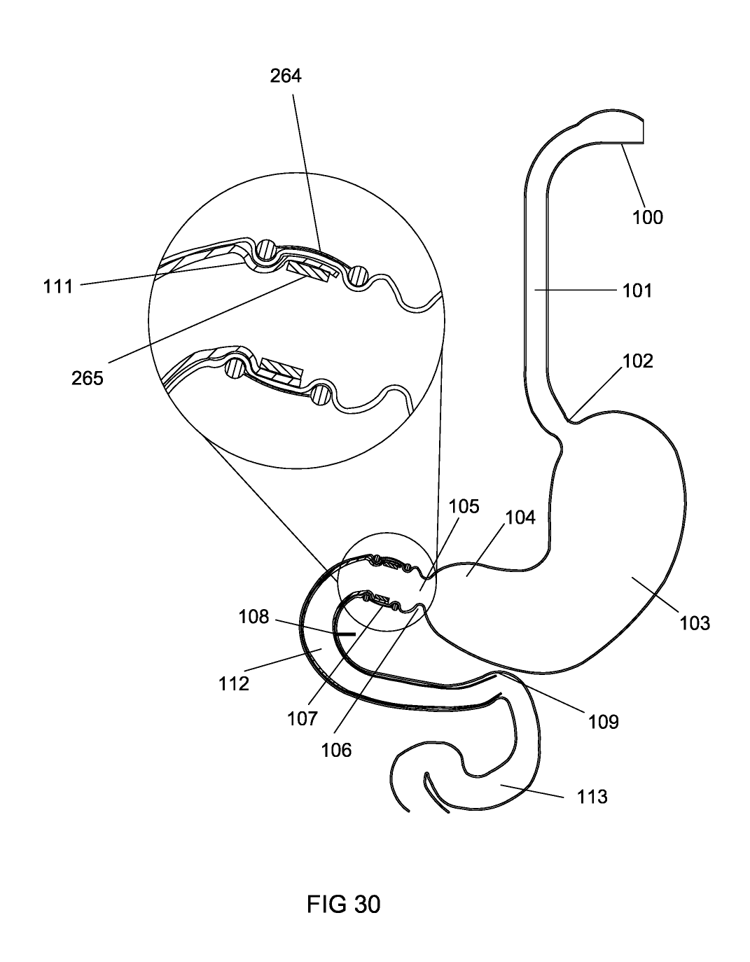

FIG. 30 is a sectional view of a portion of the digestive tract in a human body. An external band 264 is implanted around the duodenal bulb 107 and an internal tubular implant 111 is attached to the external band 264 and extended into the duodenum 112 or the jejunum 113 (e.g., to the ligament of Treitz 109). As shown, diameter interference with the external band 264 and internal implant 111 limit longitudinal movement without exerting too much radial force. Expandable ring/stent 265 is attached to internal tubular implant 111. According to exemplary embodiments, the external band 264 (of FIGS. 28-30) includes a portion or portions having a final, implanted inner diameter smaller than an outer diameter of a corresponding securing portion (e.g., the ring or stent 265) of the internal implant, such that, upon implantation, the external band 264 mates with, couples with, or otherwise interacts with the internal tubular implant 111 to prevent or resist longitudinal movement or migration of the tubular implant. Likewise, according to various embodiments, the ring of stent 265 has an expanded outer diameter of generally equal to an inner diameter of the corresponding implant location of the gastrointestinal tract. According to some embodiments, the expanded outer diameter of the stent is within about ten percent, about five percent, or about two percent of the inner diameter of the corresponding implant location of the gastrointestinal tract.

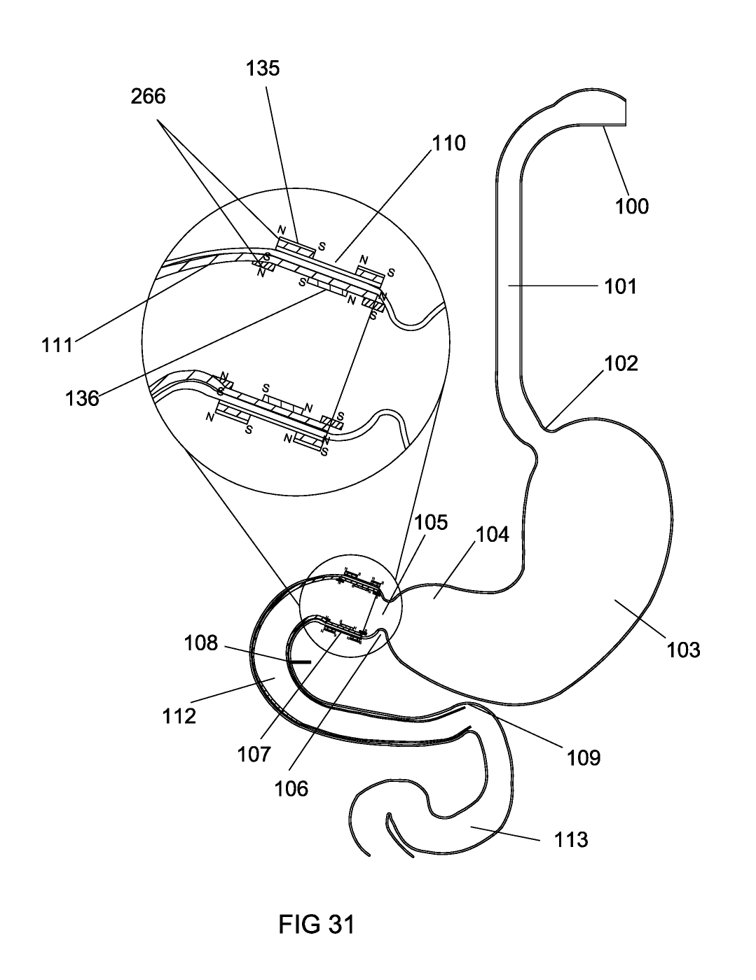

FIG. 31 is a sectional view of a portion of the digestive tract in a human body. An external band 110 is implanted around the duodenal bulb 107 and an internal tubular implant 111 is attached to the external band 110 and extended into the duodenum 112 or the jejunum 113 (e.g., to the ligament of Treitz 109). As shown, magnets 135 on the external band 110 and magnets 136 on the internal tubular implant 111 are magnetically repelled from each other and secure the internal tubular implant to the 111 to the external band implant 110. In various embodiments, the internal implant 111 also includes magnets 266, which are attracted to corresponding magnets on the external band 110.

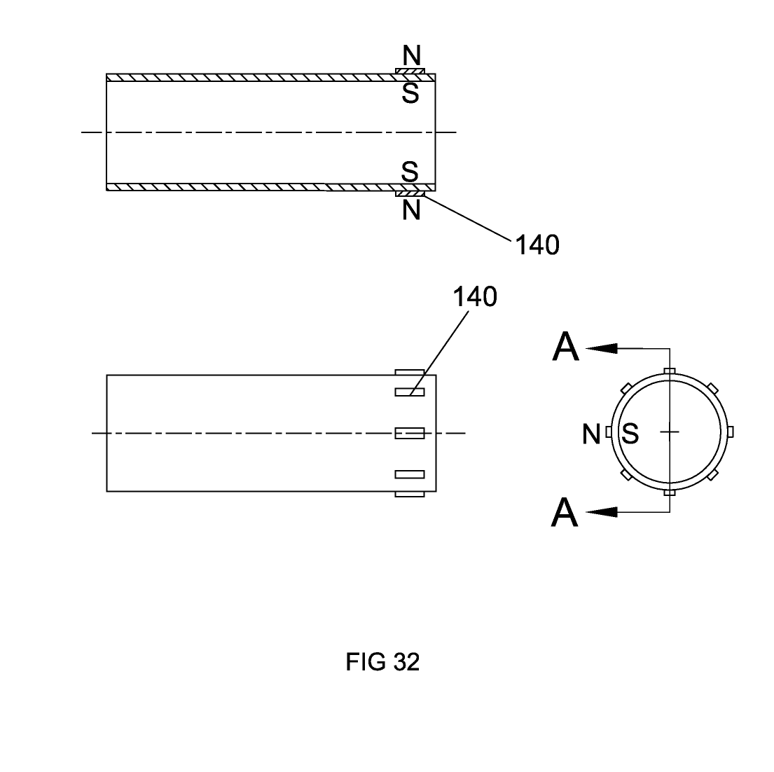

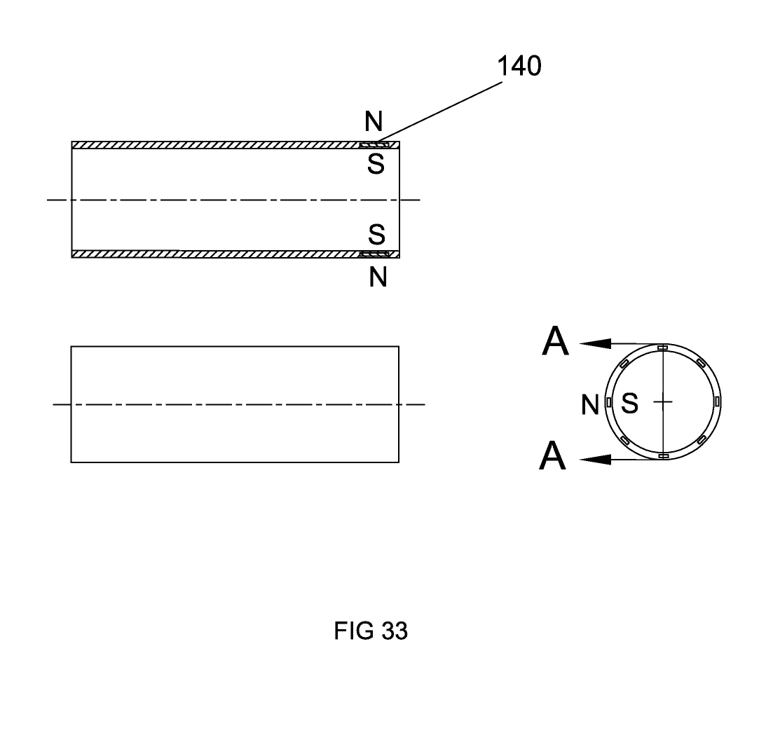

FIGS. 32-33 show various embodiments of an internal tubular implant. The tubular implant is designed to attach to another tubular implant or to an external band by a magnetic attachment means. In the embodiment of FIG. 32, the tubular implant has magnets 140 on the outside diameter. In the embodiment of FIG. 33, the tubular implant has magnets 140 in the wall thickness. In various embodiments, the magnets 140 are adapted for coupling the tubular implant to an external band, a docking element or another internal implant.

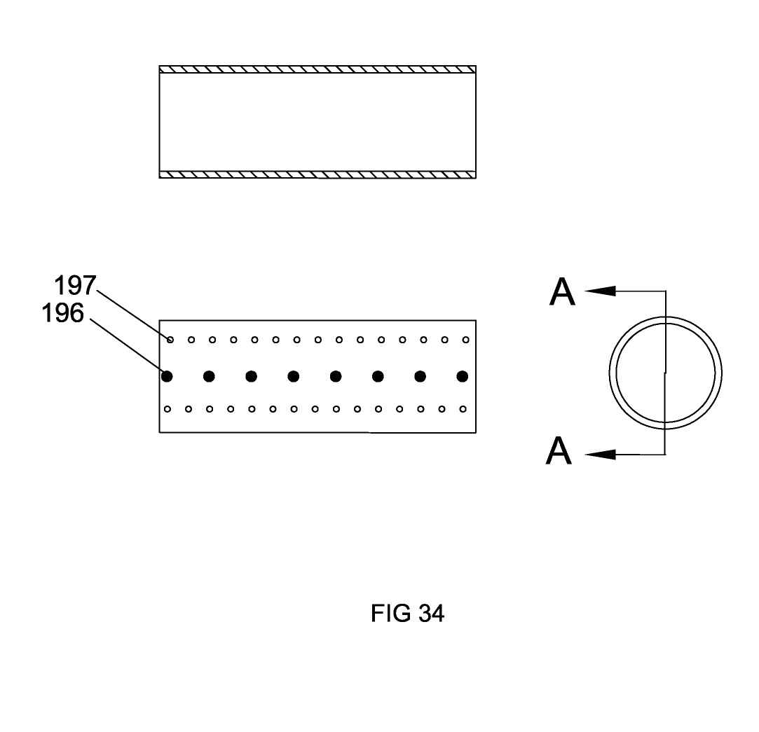

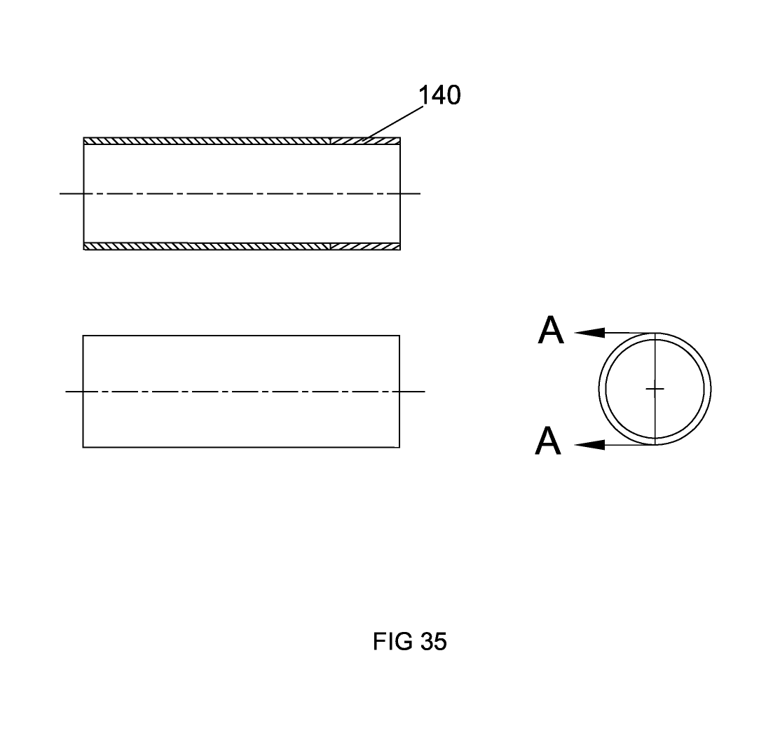

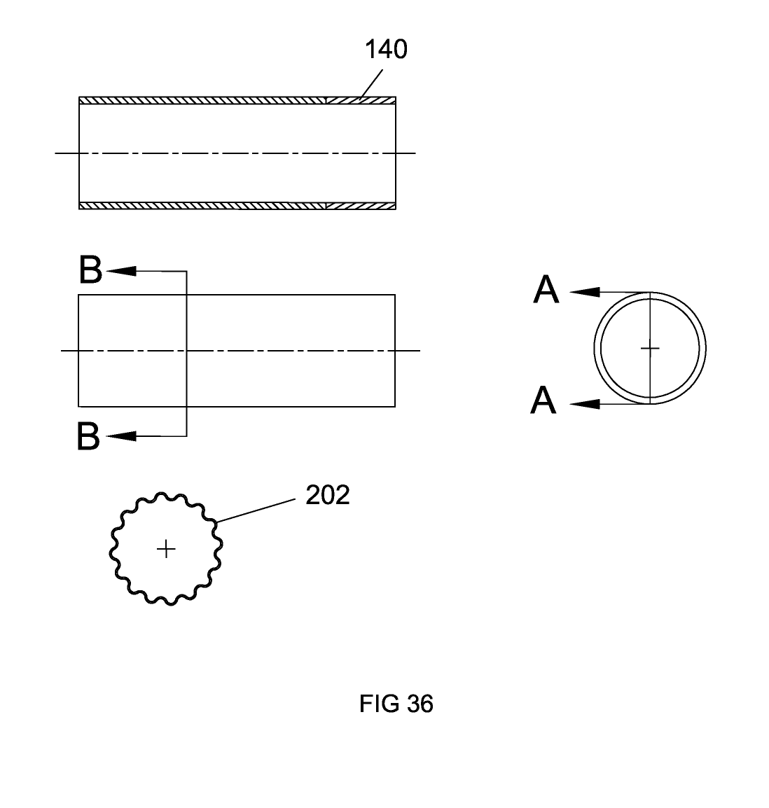

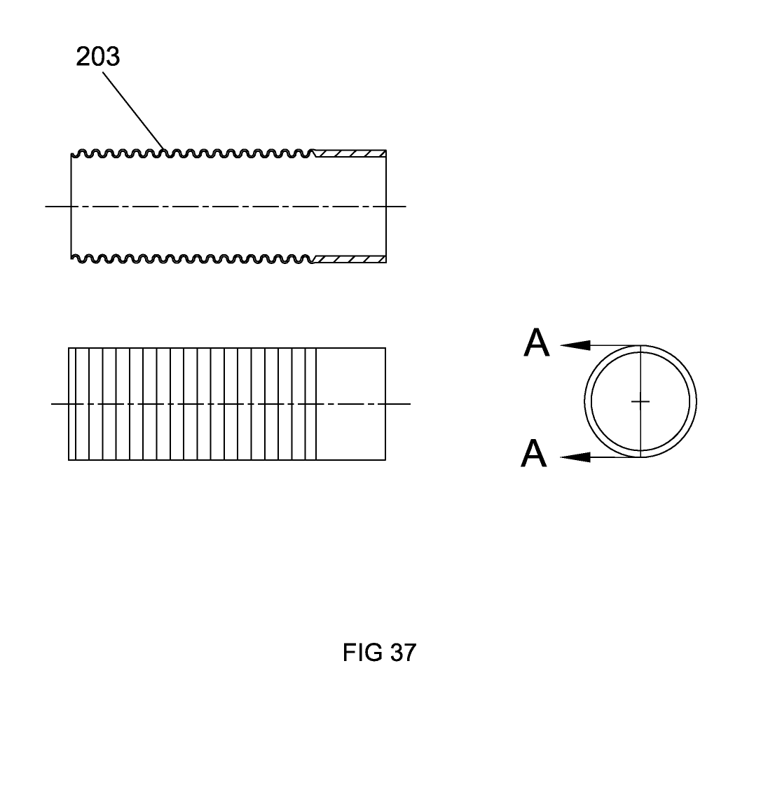

FIGS. 34-36 show various embodiments of a simple sleeve used as a component of an internal tubular implant, or for extending a tubular implant. In the embodiment of FIG. 34, the sleeve has radio-opaque markers 196 and may have holes in the sleeve 197 to allow some fluid flow through the sleeve, if required. In the embodiment of FIG. 35, the sleeve has magnetic particles or ferromagnetic material 140 incorporated into the sleeve to allow attachment of the sleeve to a magnetic docking station or tubular implant. In the embodiment of FIG. 36, the sleeve has magnetic particles or ferromagnetic material 140 incorporated into the sleeve to allow attachment of the sleeve to a magnetic docking station or tubular implant. In various embodiments, the sleeve also has longitudinal pleats 202 in the surface to allow it to collapse in diameter more uniformly and may help to reduce the loaded profile. The longitudinal pleats maybe be over the entire length or just a portion of the diameter or length. In the embodiment of FIG. 37, the sleeve has pleats around the circumference 203. These circumferential pleats will allow the tubular implant or sleeve to bend easier without kinking.

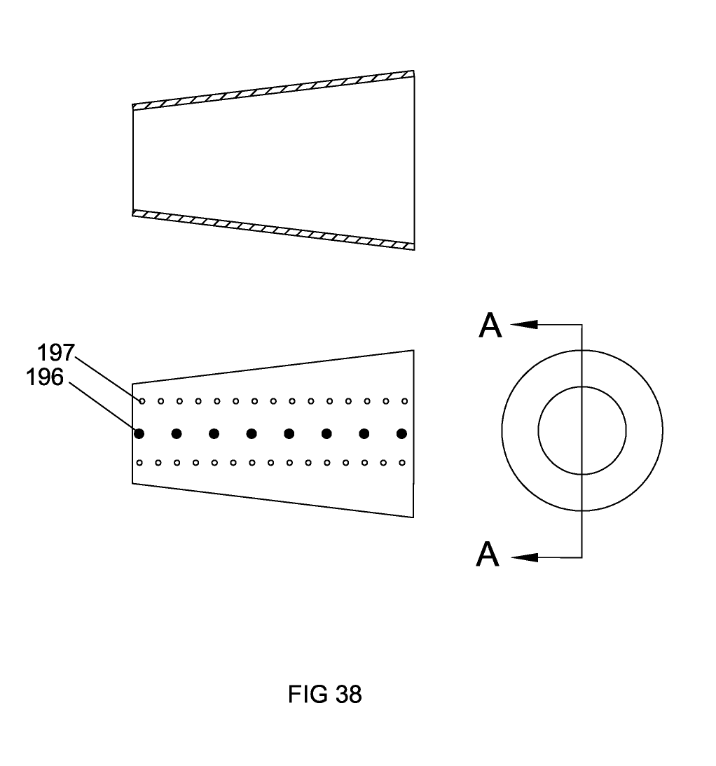

FIG. 38 is a simple sleeve with a conical diameter. The simple sleeve may be used as part of a docking station or tubular implant.

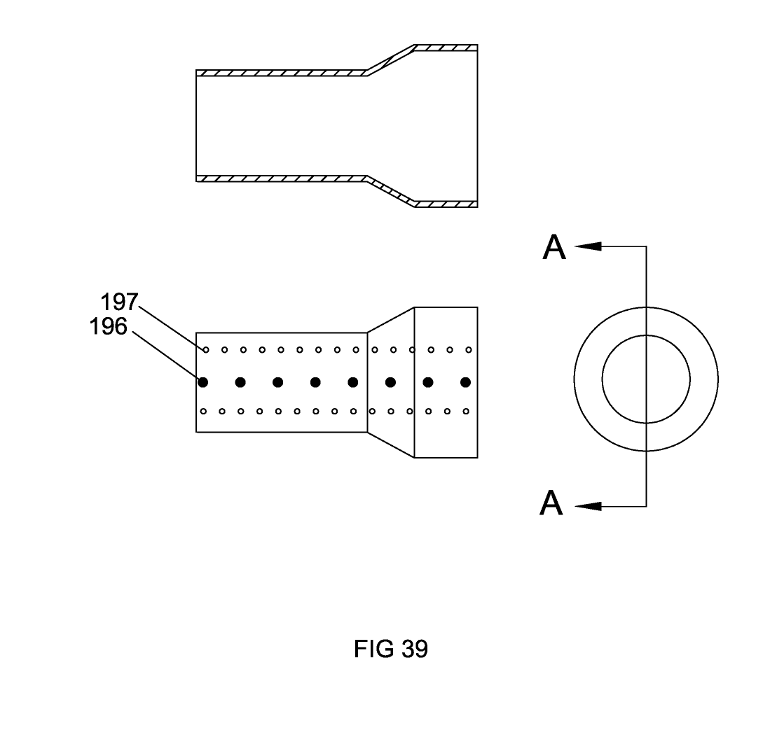

FIG. 39 is a simple sleeve with a stepped diameter. The simple sleeve may be used as part of a docking station or tubular implant.

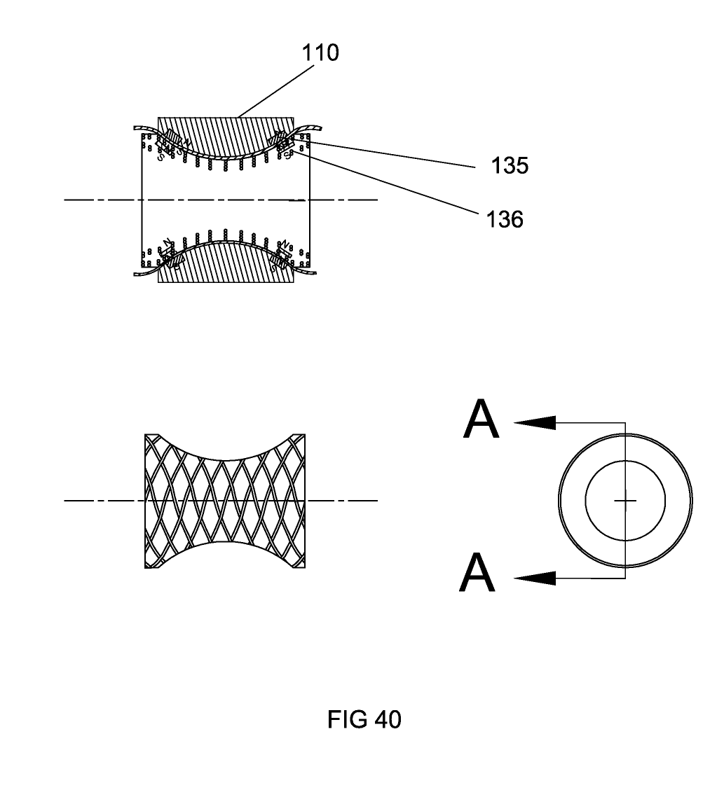

FIG. 40 is a stent that can be used to couple with an external band 110. The stent may incorporate magnets to allow magnetic attract or repulsion to an external band. As shown, magnets 136 are associated with the stent and are configured to interact with magnets 135 associated with the external band 110. According to various embodiments, the stent is integrated with or otherwise adapted to couple with an internal implant. For example, the stent may serve as a docking element for a sleeve portion of the tubular implant.

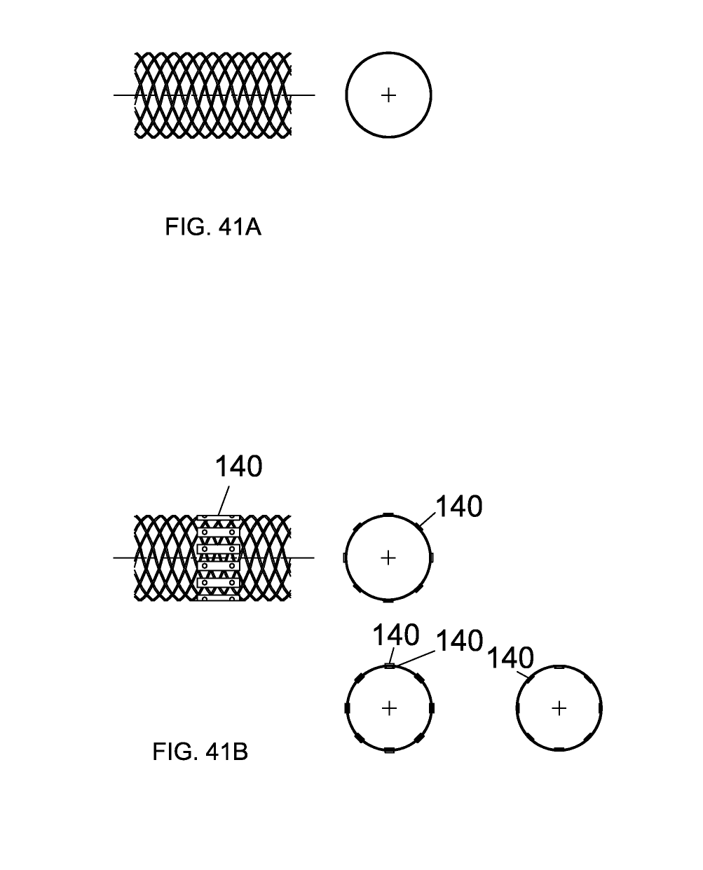

FIG. 41A shows a stent that can be used with or as a part of an internal implant. The stent can be braided from round or flat wire. The drawing of the stent is in the expanded state. The mesh of the stent may be left open or it may be covered with a suitable material previously disclosed in this application. The stent may be balloon expanded or self-expanding. The mesh of the stent may be left open or it may be covered with a suitable material previously disclosed in this application.

FIG. 41B shows a stent that can be used as a part of the internal implant. The stent can be braided from round or flat wire. The drawing of the stent is in the expanded state. The stent may include magnets 140 attached to the stent. The magnets 140 may be on the inside diameter, outside diameter, both the inside or outside diameter or incorporated into the wall. The magnets 140 can be used as a means to attach a tubular implant, such as 111. The mesh of the stent may be left open or it may be covered with a suitable material previously disclosed in this application. The stent may be balloon expanded or self-expanding. The mesh of the stent may be left open or it may be covered with a suitable material previously disclosed in this application.

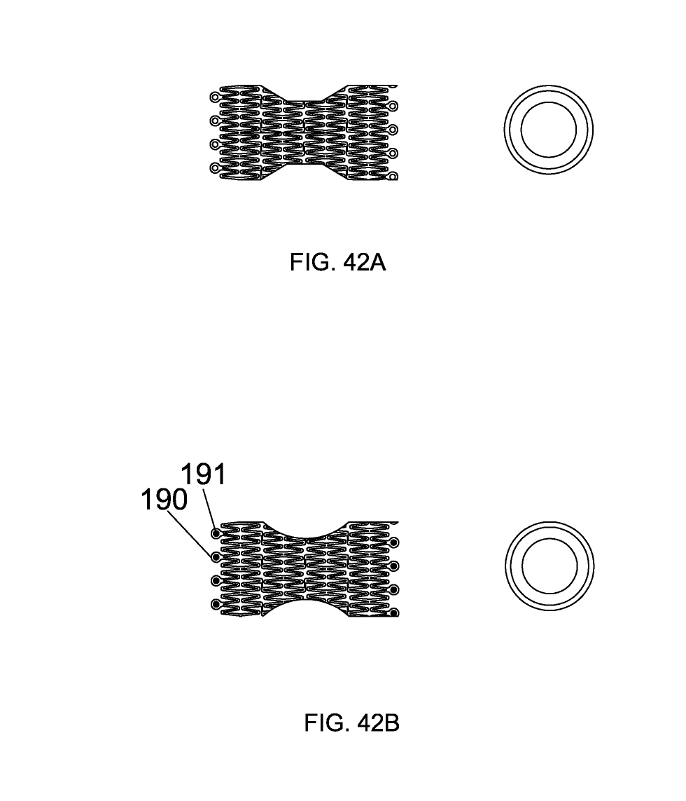

FIG. 42A shows a stent that can be used as part of an internal implant. In various embodiments, the stent is laser cut from round metal tubing or from a flat sheet of metal. The central portion of the stent's diameter may be set to a smaller diameter to provide increased resistance to stent migration. The stent may be balloon expanded or self-expanding. The mesh of the stent may be left open or it may be covered with a suitable material previously disclosed in this application.

FIG. 42B shows a stent that can be used as a part of an internal implant. According to various embodiments, the stent is laser cut from round metal tubing or from a flat sheet of metal. The central portion of the stent's diameter may be shaped into an hour glass shape to provide increased resistance to stent migration. The stent has hoops 190 at the end of the stent. The hoops may be used to interlock with a stent retainer 159 on the inner catheter 152 to prevent premature deployment before the sheath is fully retracted. Radiopaque markers 191 can be attached to the end of the stent to increase the radio-opacity of the stent. A metal insert may be pressed/swaged into the hoops 190. The insert may be made from a high atomic density material, such as tantalum, gold, platinum or iridium. The insert may take the form of a disk or sphere and may be plastically deformed to fill the hoop cavity. The stent may be balloon expanded or self-expanding. The mesh of the stent may be left open or it may be covered with a suitable material previously disclosed in this application. According to various embodiments, the stent of FIGS. 42A or 42B includes a narrow central portion adapted to generally fit within an align with the pylorus.

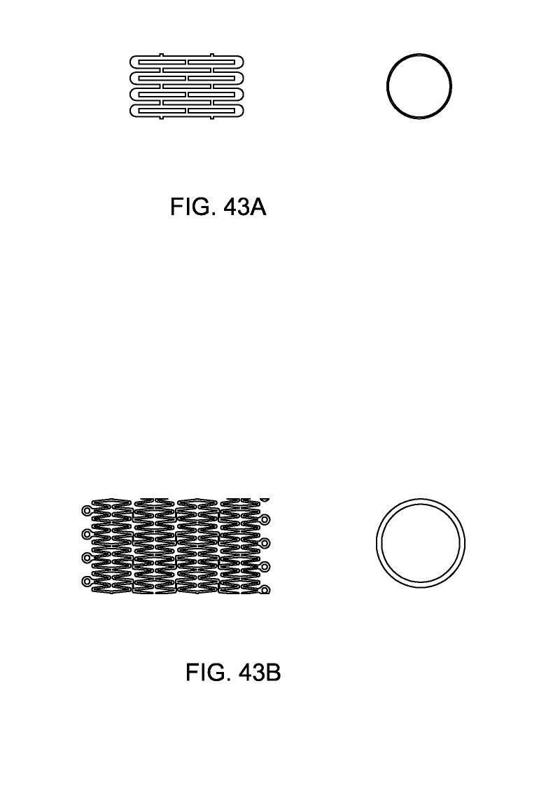

FIGS. 43A and 43B show embodiments of a stent that can be used as a part of an internal implant docking element. According to various embodiments, the stent is laser cut from round metal tubing or from a flat sheet of metal. The stent may be balloon expanded or self-expanding. The mesh of the stent may be left open or it may be covered with a suitable material previously disclosed in this application.

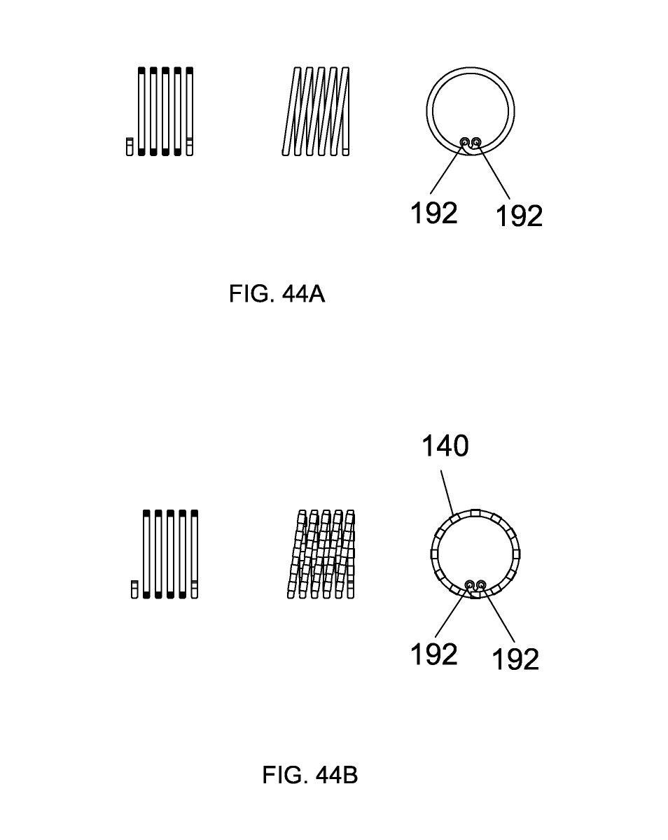

FIG. 44A shows a coil stent that can be used as a part of an internal implant. According to various embodiments, the stent is made from round or flat wire. The stent may be self-expanding or balloon expandable. The stent also may be laser cut into a coil from tubing. According to various embodiments, the stent is made from Nitinol. The mesh of the stent may be left open or it may be covered with a suitable material previously disclosed in this application. The stent has a hoop 192 at each end of the coil. The stent can be wound down onto a catheter by inserting a pin into the hoops on each end of the stent and rotating the pins in opposite directions to cause the stent to wind down onto the catheter. In the embodiment of FIG. 44B, the stent has magnets 140 on the coil of the stent. The magnets can be used as an attachment means to a tubular implant.

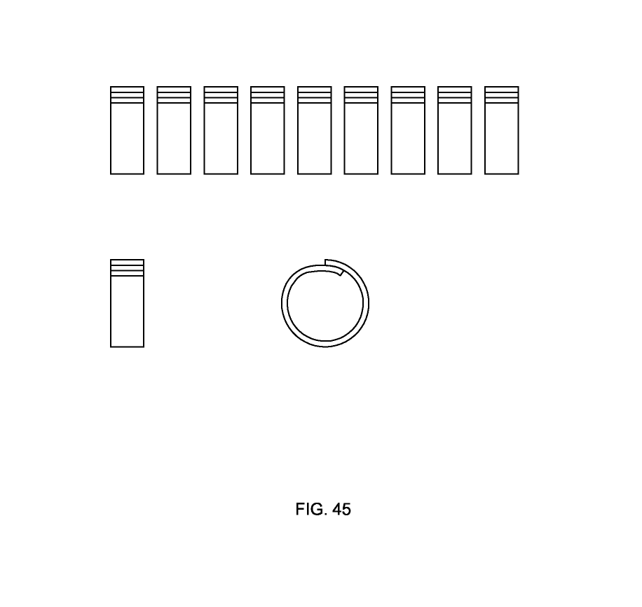

FIG. 45 shows a coil stent that can be used as a part of an internal implant. According to various embodiments, the stent is made from wire or sheet Nitinol metal. Several stents in series adjacent to each other can be used to form the docking element.

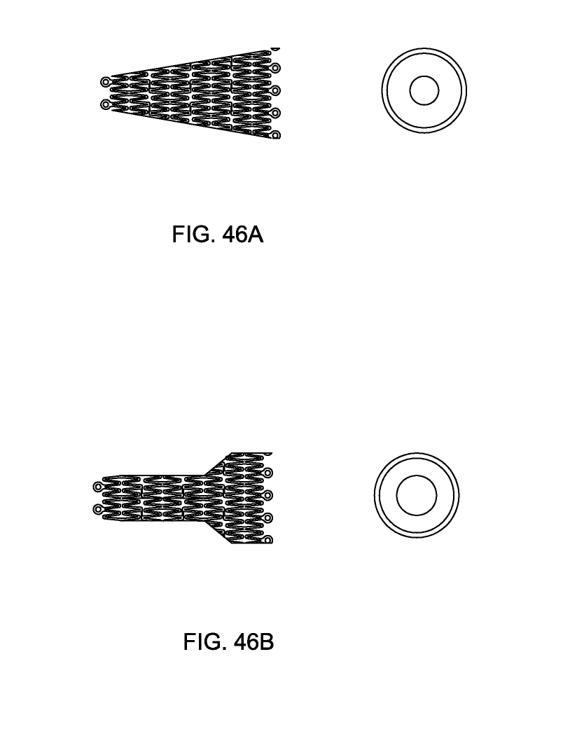

FIG. 46A shows a stent that can be used as a part of an internal implant. According to various embodiments, the stent is laser cut from round metal tubing or from a flat sheet of metal. The stent may be balloon expanded or self-expanding. The mesh of the stent may be left open or it may be covered with a suitable material previously disclosed in this application. As shown in FIG. 46A, the stent is shaped to a conical shape to provide increased resistance to stent migration and to more closely fit the anatomy. As shown in FIG. 46B, the stent is shaped to a have a stepped diameter to provide increased resistance to stent migration and to more closely fit the anatomy.

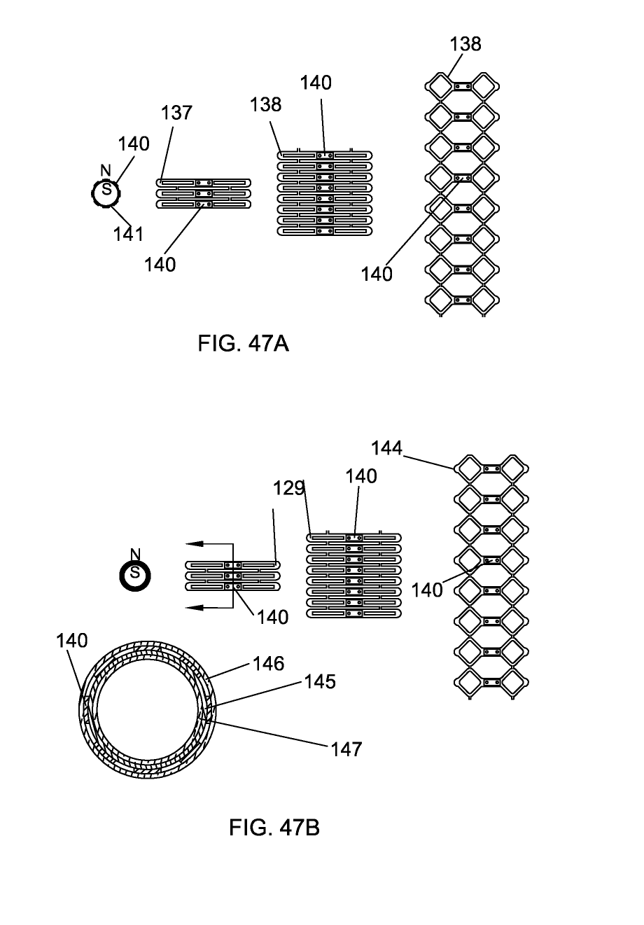

FIG. 47A shows a stent that can used as a part of an internal implant. The stents of this invention can be comprised of one or more of the following materials: Nickel titanium alloys (Nitinol), Stainless steel alloys: 304, 316L, BioDur® 108 Alloy, Pyromet Alloy® CTX-909, Pyromet® Alloy CTX-3, Pyromet® Alloy 31, Pyromet® Alloy CTX-1, 21Cr-6Ni-9Mn Stainless, 21Cr-6Ni-9Mn Stainless, Pyromet Alloy 350, 18Cr-2Ni-12Mn Stainless, Custom 630 (17Cr-4Ni) Stainless, Custom 465® Stainless, Custom 455® Stainless, Custom 450® Stainless, Carpenter 13-8 Stainless, Type 440C Stainless, Cobalt chromium alloysMP35N, Elgiloy, L605, Biodur® Carpenter CCM alloy, titanium and titanium alloys, Ti-6A14V/ELI and Ti-6A1-7Nb, Ti-15Mo tantalum, tungsten and tungsten alloys, pure platinum, platinum-Iridium alloys, platinum-nickel alloys, niobium, iridium, Conichrome, gold and gold alloys. The stent may also be comprised of one or more of the following absorbable metals: pure iron and magnesium alloys. The stent may also be comprised of the following plastics: polyetheretherketone (PEEK), polycarbonate, polyolefins, polyethylenes, polyether block amides (PEBAX), nylon 6, 6-6, 12, polypropylene, polyesters, polyurethanes, polytetrafluoroethylene (PTFE) poly(phenylene sulfide) (PPS), poly(butylene terephthalate) PBT, polysulfone, polyamide, polyimide, poly(p-phenylene oxide) PPO, acrylonitrile butadiene styrene (ABS), polystyrene, poly(methyl methacrylate) (PMMA), polyoxymethylene (POM), ethylene vinyl acetate, styrene acrylonitrile resin, polybutylene. The stent may also be comprised of the following absorbable polymers: polyglycolic acid (PGA), polylactide (PLA), poly(e-caprolactone), poly(dioxanone) poly(lactide-coglycolide).

According to various embodiments, the stent 137 stent is laser cut from a round tubing or from a flat sheet of metal. The flat representation of the stent circumference is shown in item 138. The flat representation of an expanded stent is shown in item 139. The end view of the stent is shown 141. Magnets 140 are attached to the stent on the outside diameter. The magnets 140 may be attached to the stent by use of a mechanical fastener, glue, suture, welding, snap fit or other suitable means. The stent can be either balloon expandable or self-expanding. The magnets may be located in middle of the stent or at the ends of the stent. Suitable materials for the magnets include: neodymium-iron-boron [Nd—Fe—B], samarium-cobalt [Sm—Co], alnico, and hard ferrite [ceramic] or other suitable material.

FIG. 47B shows a stent that can used as a part of an internal implant. Stent 142 may be laser cut from a round tubing or from a flat sheet of metal. The flat representation of the stent circumference is shown in item 143. The flat representation of an expanded stent is shown in item 144. The end view of the stent is shown 145. Permanent magnets 140 are attached to the stent on the outside diameter. This stent is a covered stent. The stent covering is not shown on items 142, 143 or 144. The covering are shown on the end view which shows stent 145. Stent may have an outside covering 146, inside covering 147 or both. Suitable materials for the covering include, but are not limited to: silicone, polyether block amides (PEBAX), polyurethanes, silicone polyurethane copolymers, nylon 12, polyethylene terphalate (PET), ePTFE, Kevlar, Spectra, Dyneena, polyvinyl chloride (PVC), polyethylene or polyester elastomers. The coverings may be dip coated onto the stent or they may be made as a separate tube and then attached to the stent by adhesives or mechanical fasteners, such as suture, rivets, or by thermal bonding of the material to the stent or another layer. The covering may also have drugs incorporated into the polymer to provide for a therapeutic benefit. The covering 146 or 147 may also be of biologic origin. Suitable biologic materials include, but are not limited to: Amnion, Collagen Type I, H, HI, IV, V, VI—Bovine, porcine, ovine, placental tissue or placental veins or arteries and small intestinal sub-mucosa.

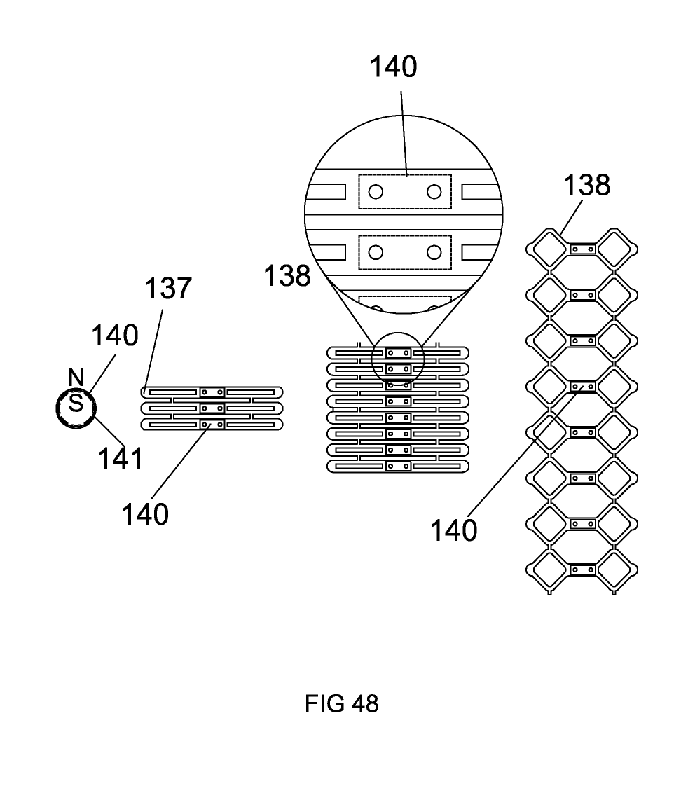

FIG. 48 shows a stent that can used as a part of an internal implant. Stent may be laser cut from a round metal tubing or from a flat sheet of metal. The flat representation of the stent circumference is shown in item 138. The flat representation of an expanded stent is shown in item 137. The end view of the stent is shown 141. Magnets 140 are attached to the stent on the inside diameter. The magnets may be attached to the stent by use of a mechanical fastener, glue, suture, welding, snap fit or other suitable means. The stent can be either balloon expandable or self-expanding. The magnets may be located in middle of the stent or at the ends of the stent. Suitable materials for the magnets include: neodymium-iron-boron [Nd—Fe—B], samarium-cobalt [Sm—Co], alnico, and hard ferrite (ceramic) or other suitable material. The stent may be balloon expanded or self-expanding

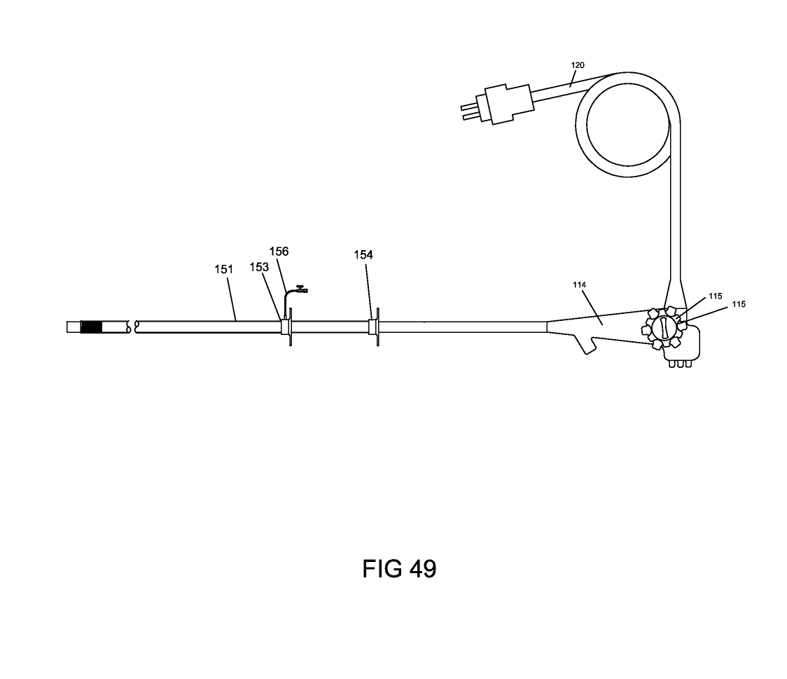

FIG. 49 shows the delivery catheter for the apparatus disclosed loaded over an endoscope.

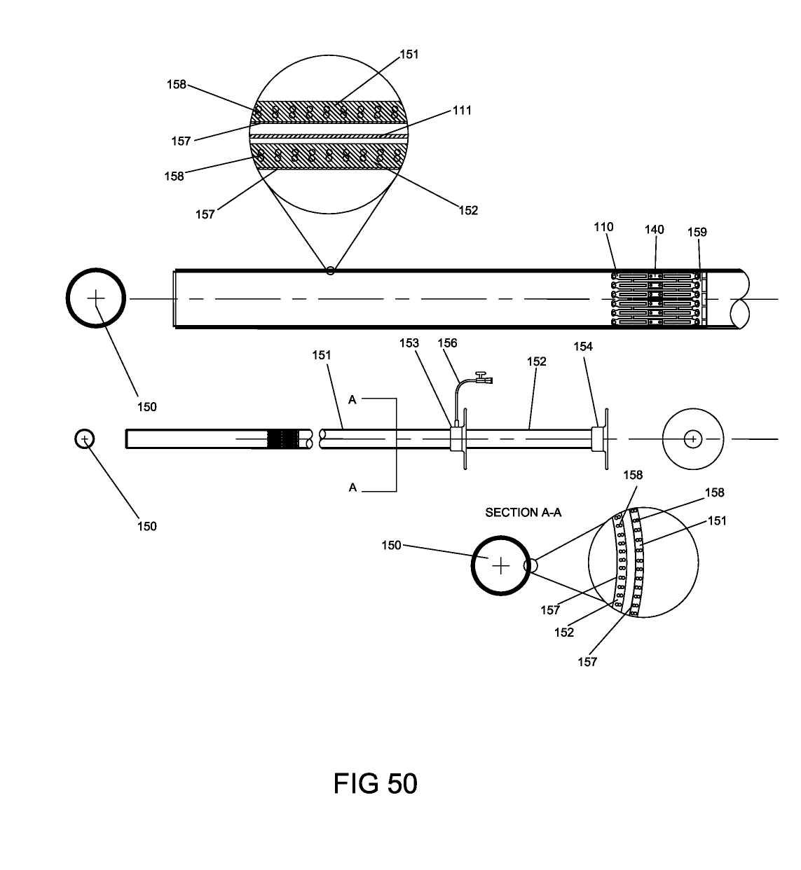

FIG. 50 shows an alternative embodiment drawing of a delivery catheter for a self-expanding internal tubular implant. The tubular implant is located distal to the docking element. The delivery catheter could also be used for delivery of a stented sleeve construct where the sleeve and stent are integrated together into one implant. The delivery catheter is constructed with a central lumen 150 large enough to allow the catheter to be loaded over the outside diameter of the endoscope 114. The delivery catheter consists of an outer catheter 151 and an inner catheter 152.

To load the tubular implant onto the delivery catheter the outer sheath handle 153 is retracted towards the inner catheter handle 154 until distance is as small as possible. The outer sheath is then partially closed by advancing the outer sheath handle 153 away from the inner sheath handle 154. Continue advancing the outer sheath 151, when the tubular implant is completely covered by the outer sheath 151, the loading process is complete for the tubular implant. The delivery catheter also has a space on the inner catheter for the modular implant to be loaded. Attached to the inner catheter is a stent retainer 159. The purpose of the stent retainer 159 is to prevent the stent from releasing from the delivery catheter prematurely during deployment. The stent retainer 159 is fastened to the inner catheter. The stent retainer 159 can be made from metal or plastic and can be made radio-opaque by making it from a radio-opaque material such as tantalum. The stent retainer 159 has a complementary shape that holds the tips on the stent and does not allow the stent to move distally or forward until the outer sheath 151 is fully retracted to the stent retainer 159. The catheter has a side port 156 which allows the space between the inner and outer sheaths to be flushed with saline. The outer sheath 151 and inner sheath 152 may be made from a simple single layer polymer extrusion, such as from polyethylene or PTFE. The outer sheath 151 may also be constructed in the following manner. The sheath inner diameter surface is constructed of a thin wall PTFE liner 157. A layer of reinforcement 158 is placed over the PTFE liner 157. According to various embodiments, the reinforcement is either a braid of wire or a coil of wire. The wire cross-section can be either round or rectangular. In some embodiments, the wire is made from a metal such as 316, 304 stainless steel, Nitinol, or other suitable material. The wire diameters are typically in the 0.0005 inch to 0.010 inch diameter range. The outer jacket material may be reflowed into the reinforcement layer by melting the material and flowing the melted polymer into the spaces in between the braided wire or the coiled wires.

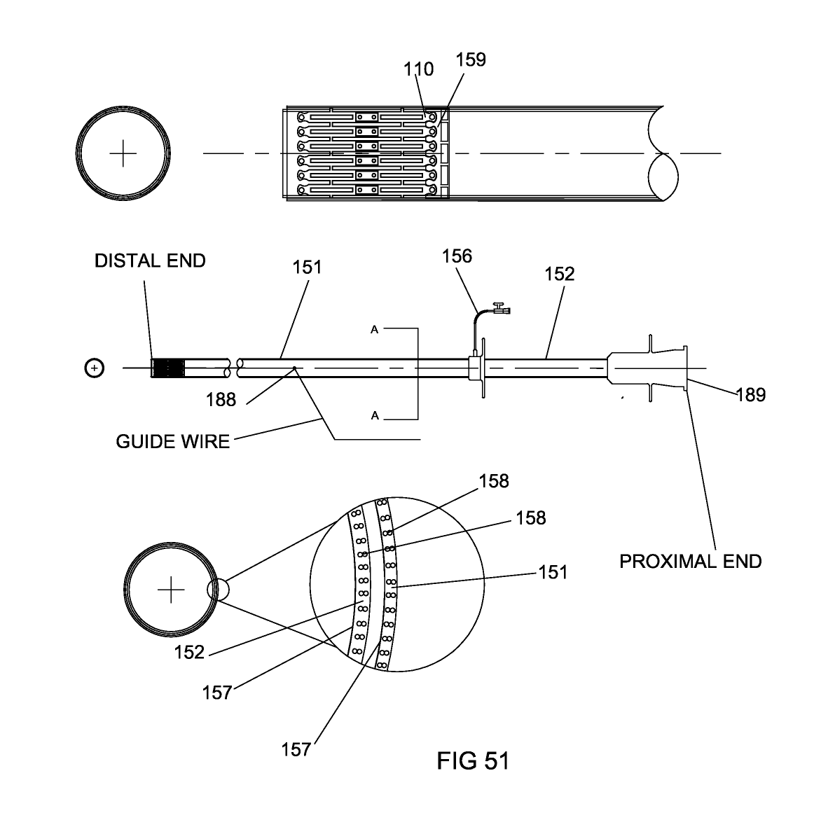

FIG. 51 shows an alternative embodiment of a delivery catheter for a self-expanding internal tubular implant or for both 110 and 111 on the same catheter. The delivery catheter is constructed with a smaller outside diameter to allow the catheter to be inserted through the working channel of the endoscope 114. The delivery catheter consists of an outer catheter 151 and an inner catheter 152. Attached to the inner catheter is a stent retainer 159. The purpose of the stent retainer 159 is to prevent the stent from releasing from the delivery catheter prematurely during deployment. The stent retainer 159 is fastened to the inner catheter. The stent retainer 159 can be made from metal or plastic and can be made radio-opaque by making from it from a radio-opaque material such as tantalum. The stent retainer has a complementary shape that holds the tips on the stent and does not allow the stent to move distally or forward until the outer sheath 151 is fully retracted to the stent retainer 159. The catheter has a side port 156 which allows the space between the inner and outer sheaths to be flushed with saline. The outer sheath 151 and inner sheath 152 may be made from made from a simple single layer polymer extrusion such as from polyethylene or PTFE. The outer sheath 151 may also be constructed in the following manner. The sheath inner diameter surface is constructed of a thin wall PTFE liner 157. A layer of reinforcement 158 is placed over the PTFE liner 157, the reinforcement may be either a braid of wire or a coil of wire. The wire cross-section can be either round or rectangular. According to various embodiments, the wire is made from a metal such as 316 or 304 stainless steel or Nitinol or other suitable material. The wire diameters are typically in the 0.0005 inch to 0.010 inch diameter range. The outer jacket material may be reflowed into the reinforcement layer by melting the material and flowing it into the spaces in between the braided wire or the coil wires. The outside diameter of this catheter will range typically from 1 mm to 4 mm. The catheter can be constructed to be an over the wire catheter or a rapid exchange catheter. For a rapid exchange design, the guidewire will enter the central lumen of the distal end of the catheter and exit at point 188. For an over the wire catheter design, the guidewire will enter the central lumen of the distal end of the catheter and exit at point 189.

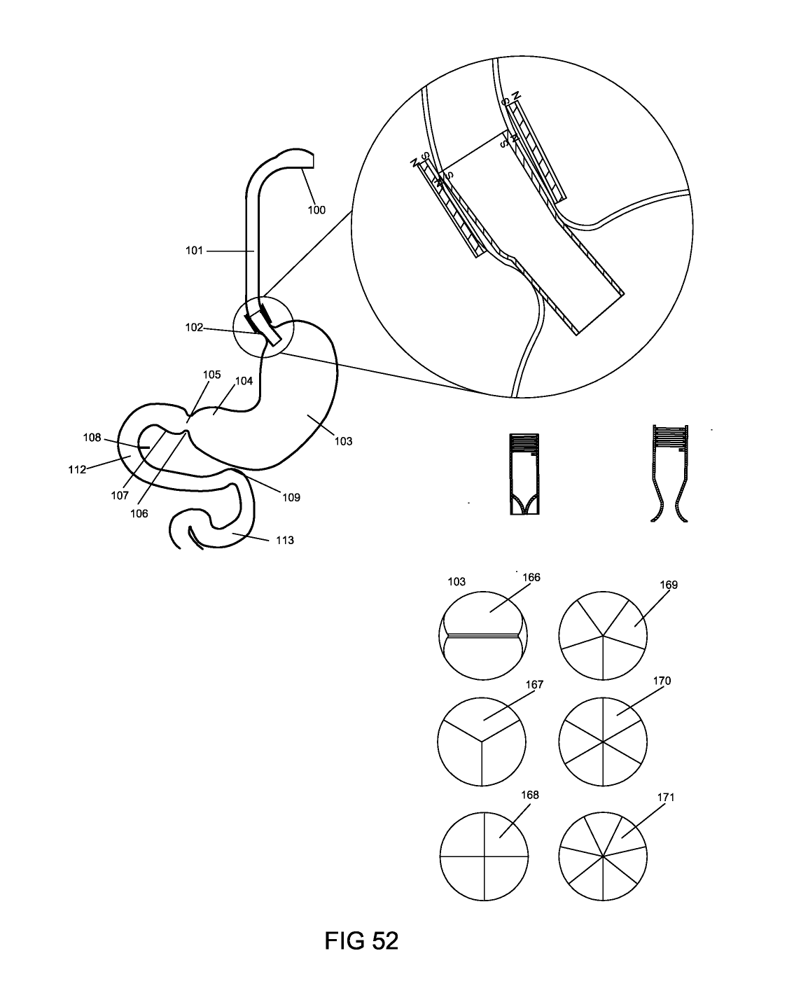

FIG. 52 is a cross-sectional view of a portion of the digestive tract in the body. An external band is implanted in the esophagus at gastro-esophageal junction 102. An internal tubular implant is attached to the external band. The tubular implant can have bi-leaflet anti-reflux valve 166, a tri-leaflet anti-reflux valve 167, a quad-leaflet anti-reflux valve 168, a penta-leaflet anti-reflux valve 169, a six-leaflet anti-reflux valve 170 or seven-leaflet anti-reflux valve 171. The implant can also be a stoma.

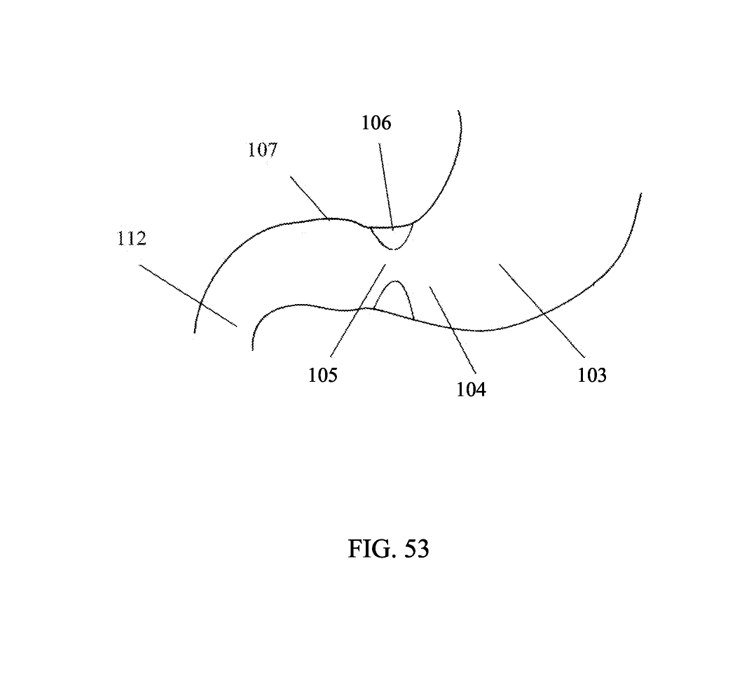

FIG. 53 is a schematic, sectional view of a portion of a human digestive tract. As a person ingests food, the food enters the mouth, is chewed, and then proceeds down the esophagus (not shown) and into the stomach 103. The food mixes with enzymes in the stomach 103. The stomach 103 converts the food to a semi-fluid substance called chyme. The chyme enters the pyloric antrum 104 and exits the stomach 103 through the pylorus 106 and pyloric orifice 105. The small intestine is about 21 feet long in adults and is comprised of three sections: the duodenum 112, the jejunum (not shown) and the ileum (not shown). The duodenum 112 is the first portion of the small intestine and is typically 10-12 inches long. The duodenum 112 is comprised of four sections: the superior, descending, horizontal and ascending duodenum. The duodenum 112 ends at the ligament of Treitz. The duodenal bulb 107 is the portion of the duodenum which is closest to the stomach 103 and generally has diameters ranging from 35 mm to 55 mm in humans.

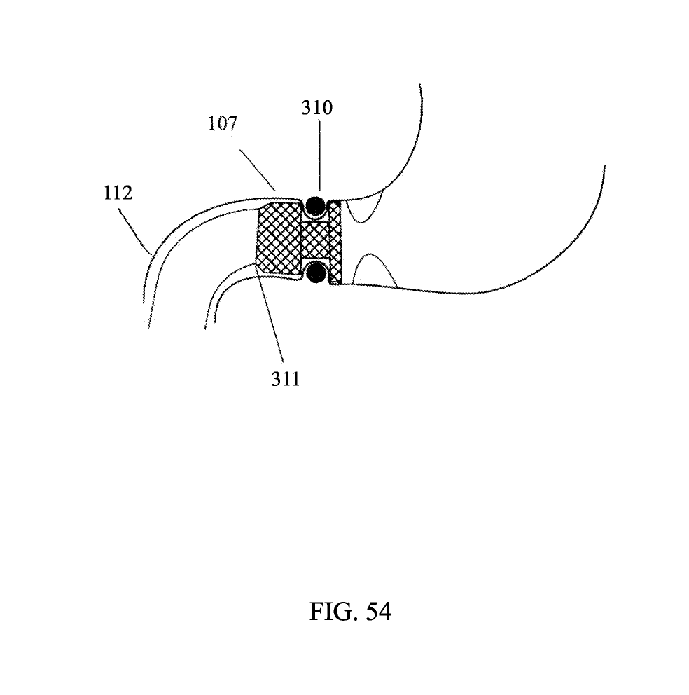

As shown in FIG. 54, in a sectional view, an external implant 310 is implanted around the duodenal bulb 107 and an internal tubular implant 311 is coupled to the external implant 310 by mechanical anatomical interlocking and extended into the duodenum 112. The specific shape and mechanical characteristics of the external band 310 (in this instance a semi-rigid ring with a circular cross-section) and the response of the duodenal bulb tissue to the placement of the external implant 310 create a mechanical interlocking feature on the inner surface of the duodenum to secure the internal tubular implant 311 to the external implant 310 in a removable or reversible configuration.

In various embodiments, the external implant 310 and the internal tubular implant 311 anchor by coupling with each other using any of a variety of techniques including, for example anchoring means that are based on mechanical interference, elasticity, spring force, shape memory transformation, etc.

According to various embodiments, the internal tubular implant 311 is configured such that it exerts little radial force against an internal surface of the gastrointestinal tract. Those familiar with gastro-intestinal medical devices will understand these internal tubular implants, like 311, are not designed and sized to behave like traditional stents whose primary design goal is to exert radial force on tissue to either prop a lumen open or otherwise help the stent anchor. Also, according to various embodiments, the external implant 310 is sized and shaped such that, in its final, implant configuration, it exerts little radial force against an external surface of the gastrointestinal tract. It is the combination of the shape of the internal implant 311, the shape of the external implant 310 and the response of the tissue in the region they are implanted that allows for the simple but effective interlocking of components for safe anchoring and prevention of migration of the internal implant 311 by the forces of peristalsis. For example, in embodiments, such as shown in FIG. 54, the external band 310 has an implanted inner diameter generally smaller than the outer diameter of the duodenal bulb 107 and slightly deforms it. This creates an anatomical ridge on the inner surface of the duodenum 112. The internal implant 311 has an hour-glass shape, the waist of which interlocks with the anatomical ridge without the external 310 and internal 311 implants exerting excessive forces on the tissue.

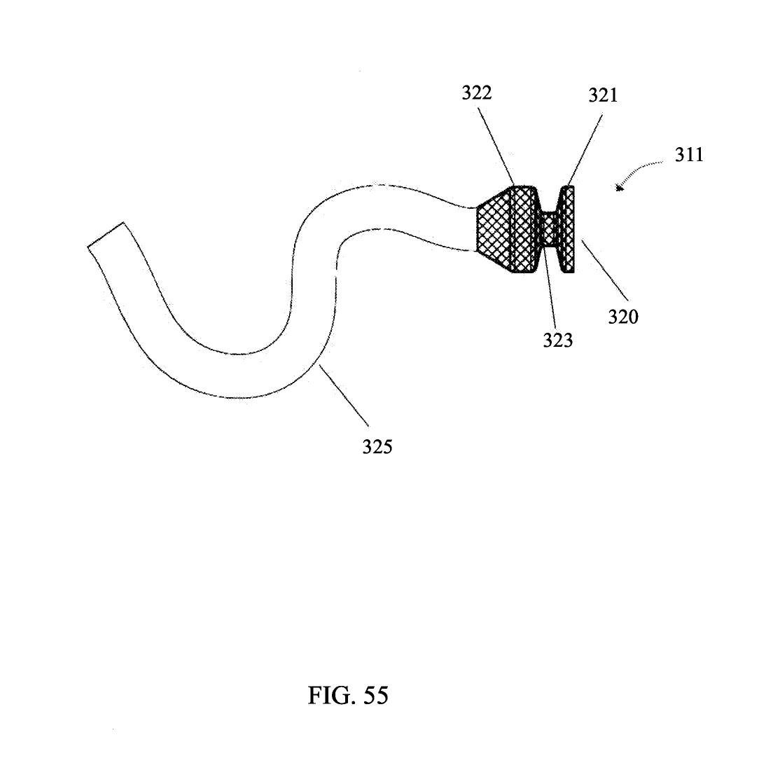

FIG. 55 shows in more detail the construction of the internal implant 311 shown in FIG. 54. The proximal body of the implant 320 has an hour glass shape with the diameters of the top and bottom flanges (321 and 322) generally between 30 mm and 50 mm in diameter and the waist area of the implant 323 having a diameter generally between 10 and 30 mm respectively. It is constructed of materials that possess memory for shape and thus the implant can be fitted in to a small cylindrical capsule of a diameter less than 15 mm and then expanded to its final net hour-glass shape upon delivery to its desired location. This allows the physician to place and remove the internal implant 311 under endoscopic guidance without having to perform surgery. The implant 311 can be constructed from materials such as a nickel titanium alloy and the preferred method of construction would be a wire braided or laser cut structure that exhibits memory for shape. Alternatively, it is also conceivable that the implant 311 could be constructed of flexible elastomers that can be form fitted in to a small capsule and would open up to the desired hour glass shape upon released from the delivery capsule. The implant 311 in FIG. 55 may be covered with suitable fabrics or polymeric films or could be coated with such materials. Examples of materials used to cover or coat the implant 311 would be polyurethanes, silicones and fluoro-polymers, such as ePTFE.

Attached to the proximal hour glass shaped anchor portion 320 of the implant 311 is a thin flexible polymer sleeve 325 between 0.001 in. to 0.005 in. thick and with a length of between 10 inches and 50 inches so that it extends in to the proximal jejunum past the ligament of Treitz. The sleeve 325 allows food to pass through its internal lumen by the natural peristaltic motion of the intestine but the impermeable sleeve 325 prevents absorption of nutrients and neuro-hormonal signaling associated with contact of food with the first part of the small intestine. However, the sleeve 325 still allows for passage of bile, pancreatic juice and other secretions to flow around it.

The sleeve 325 is preferably constructed of materials that can resist both the highly acidic stomach juice as well as the highly alkaline bile. The sleeve 325 could be constructed of, for example, fluoro-polymers, such as ePTFE, PFA, Modified PTFE, PVDF and ETFE or certain chemical resistant polyurethanes.

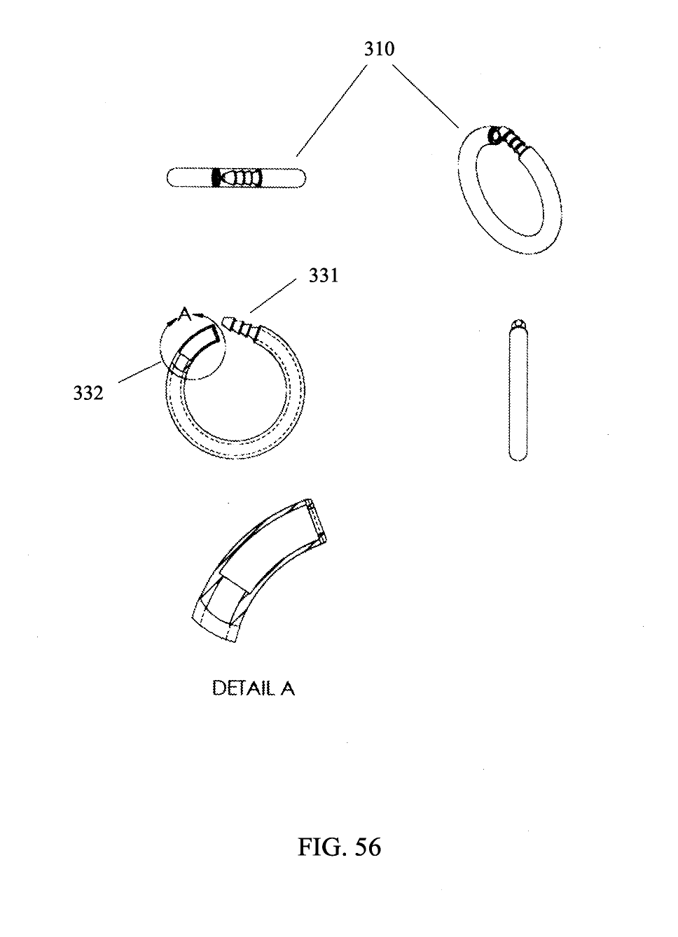

FIG. 56 shows in more detail the construction of the external implant 310 shown in FIG. 54. The implant 310 consists of a ring-shaped element with a circular cross-section that is flat when open but can be closed and locked in various positions to create different diameter rings with a barb shaped end 331 on one side that mates with a hollow tubular section 332 on the other end. Implant 310 is constructed of materials that can be fitted into a small cylindrical trocar used during laparoscopic surgery of a diameter less than 15 mm and then configured to its final net shape upon delivery to its desired location. This allows the physician to place and remove the external implant 310 under laparoscopic guidance. The implant 310 can be constructed from materials such as a silicone or polyurethane.

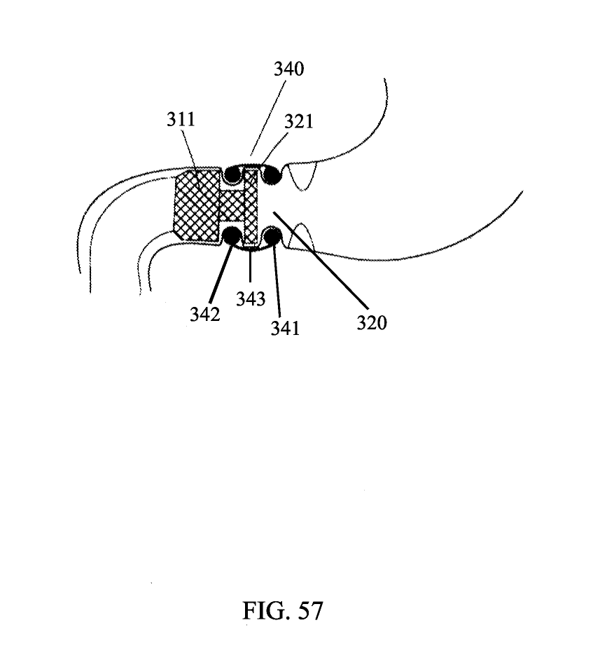

FIG. 57 shows a cross-sectional view of a band shaped external implant 340. The band 340 consists of two ring-shaped members with a circular cross-section that are connected by an element with a thin flat cross-section. The implant 340 interlocks with the same hour-glass shaped proximal end 320 of implant 311 as shown in FIG. 55 and described earlier except that the inter-locking happens with the upper flange 321 of the internal implant 311.

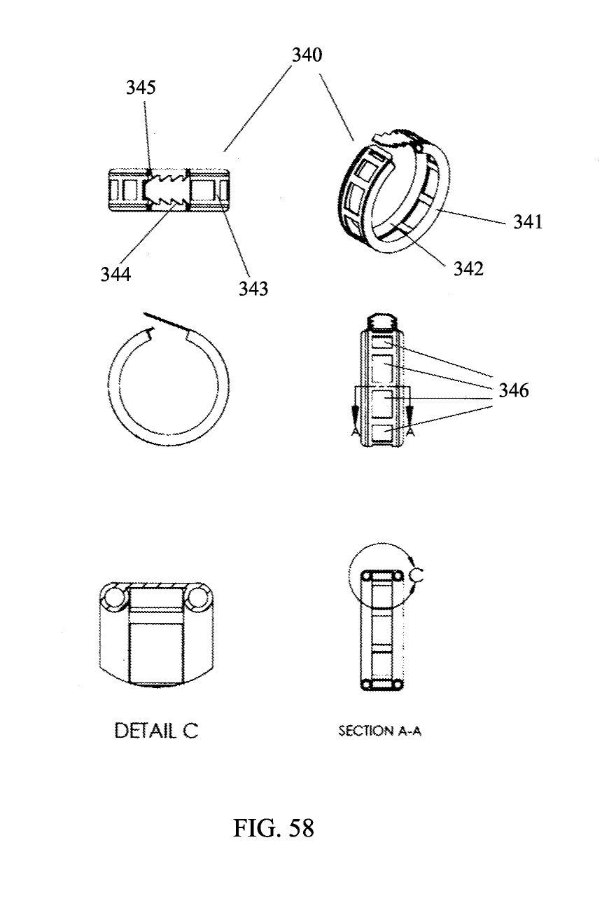

FIG. 58 shows in more detail the construction of the external implant 340 shown in FIG. 57. The band shaped implant consists of two ring shaped members 341 and 342 with a circular cross-section that are connected by an element with a thin flat cross-section 343. The implant is flat when open so that it can be inserted through long trocars but can be closed and locked in various positions to create different diameter rings with a Christmas tree shaped end on one side 344 that mates with a slotted section 345 on the other end. The implant 340 also has several slots 346 in the flat section 343 of the implant 340. These slots 346 are meant to allow the duodenal tissue to extrude out of the openings so as to create additional positive anchoring when the implant 340 interlocks with the internal implant 311 of FIG. 55. The external band 340 is constructed of materials that can be fitted into a small cylindrical trocar used during laparoscopic surgery of a diameter less than 15 mm and then configured to its final net shape upon delivery to its desired location. This allows the physician to place and remove the external implant 340 under laparoscopic guidance. The implant 340 can be constructed from materials such as a silicone or polyurethane.

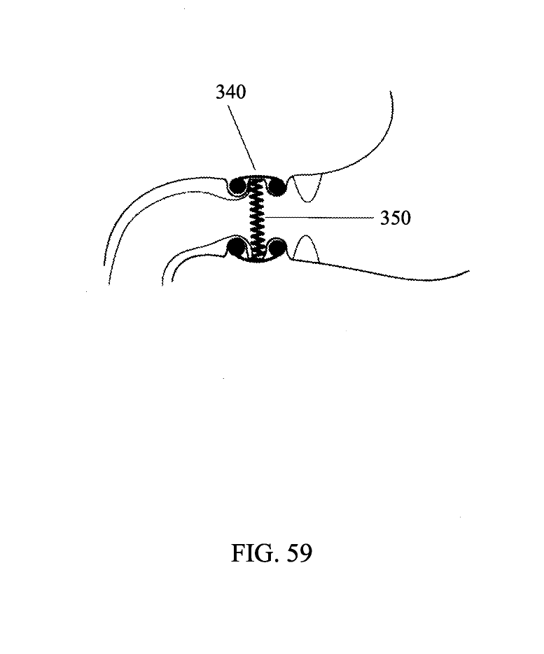

FIG. 59 shows the same external band implant 340 of FIG. 58 placed around the duodenal bulb but with a different single flanged internal implant 350 coupling with the external band 340. The implant 350 exerts a spring force on the tissue allowing it to extrude through the slots 346 (see FIG. 58) for interlocking and safe anchoring of the implant 350.

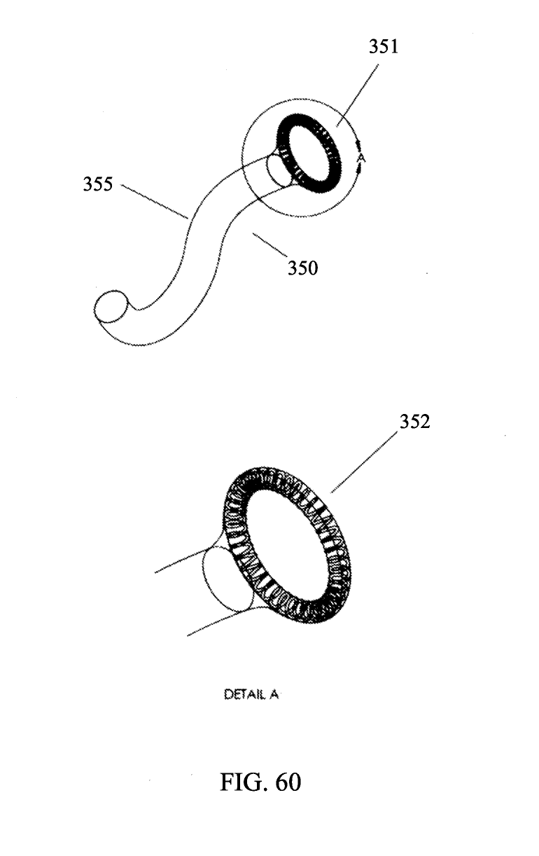

In FIG. 60, the internal implant 350 with a single proximal spring flange 351 is shown in detail. Internal implant 350 consists of a metal or polymer spring which is designed to exert low but positive radial force on the duodenal tissue when expanded to its full diameter. This slightly deforms the tissue and allows it to extrude through the slots 346 (see FIG. 58) of the external band 340. The fully open diameter of the internal implant 350 is preferably 3 to 15 mm larger than the smallest internal diameter of the external band 340 allowing for positive interlocking of the external band 340 with the internal implant 350. In order to be able to collapse the spring for implant placement and withdrawal, a drawstring 352 is threaded through the implant and serves as the mechanism for opening and closing of the spring flange. Pulling on the drawstring causes the spring windings to come closer together and this can be achieved by either hooking or grasping the drawstring with appropriate endoscopic instruments. The sleeve portion of the implant 355 is almost identical to the sleeve 325 shown in FIG. 55 in construction except it needs to be connected to the spring flange in a different fashion. The spring flange is also preferably covered with a jacket of a low friction material such as ePTFE to prevent necrosis and tissue damage.

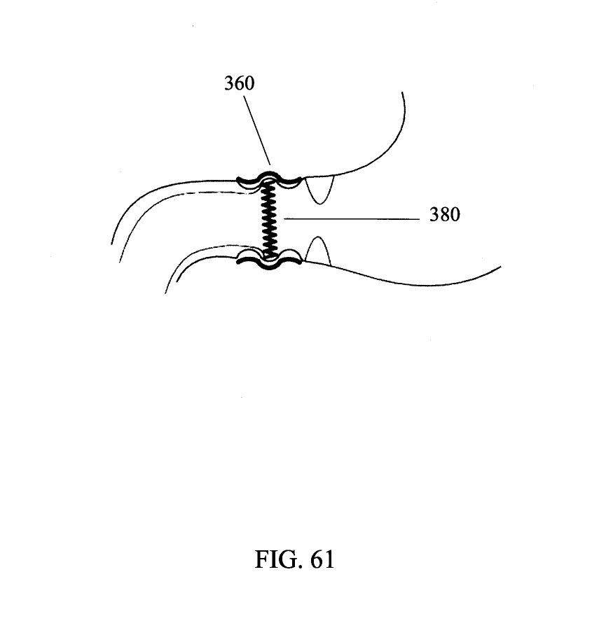

In FIG. 61, the internal implant 350 is anchored with an external band 360 of a different design. The anchoring and interlocking principle is the same as before with the spring flange exerting a small radial force on the tissue allowing it to interlock with certain design features of the external implant 360.

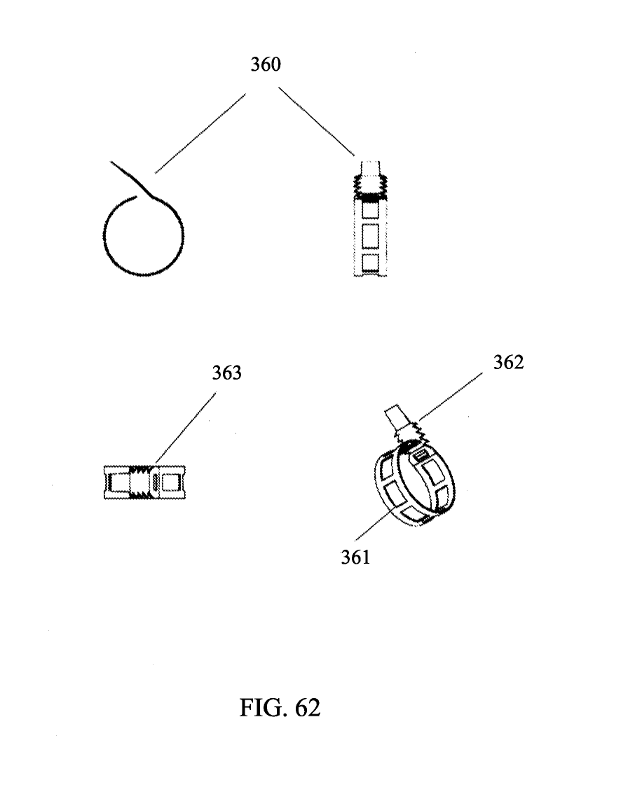

In FIG. 62, the construction of the external implant 360 is shown in more detail. The band-shaped implant is preferable made from a flat sheet of elastomeric material such as silicone or polyurethane though could be constructed of metals with articulating hinges like in a watch in order for the implant to be able to encircle the duodenal bulb upon implantation and exert a small inward radial force to slightly distend the duodenal tissue once locked to a certain inner diameter with the help of the Christmas tree-shaped locking end 362 that can be threaded through a slot on one end of the band 363. Larger slots 361 are provided on the central longitudinal axis of the band to allow tissue to extrude through by the force exerted by the slightly large internal implant similar to the external implant shown in FIG. 57.

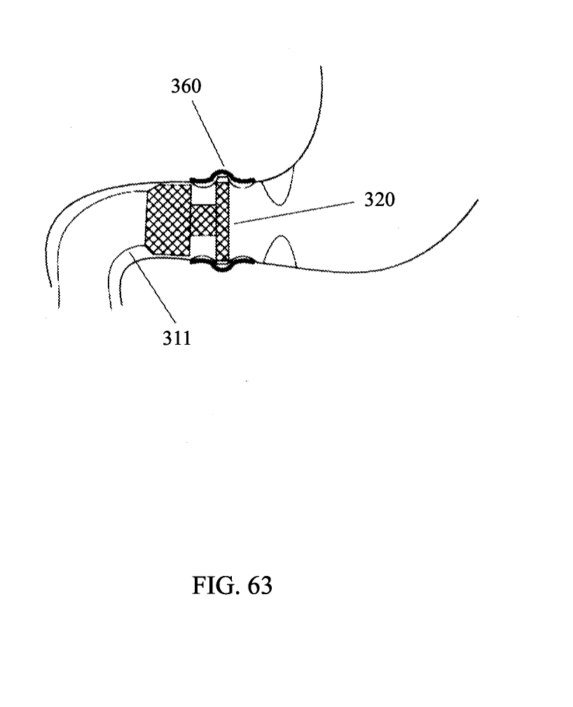

It will be clear to someone familiar with medical technology that this concept of interlocking an internal implant placed in the duodenum with an external band can be implemented with various combinations of designs for the internal and external components and similar such designs as described above. For example, in FIG. 63, a combination of the external band 360 shown in FIG. 62 with the internal implant design 311 with a proximal hour-glass shape end 320 as shown in FIG. 54 is shown.

In this embodiment, the external band has an implanted inner diameter generally equal to an outer diameter of the desired mounting location of the gastrointestinal tract.

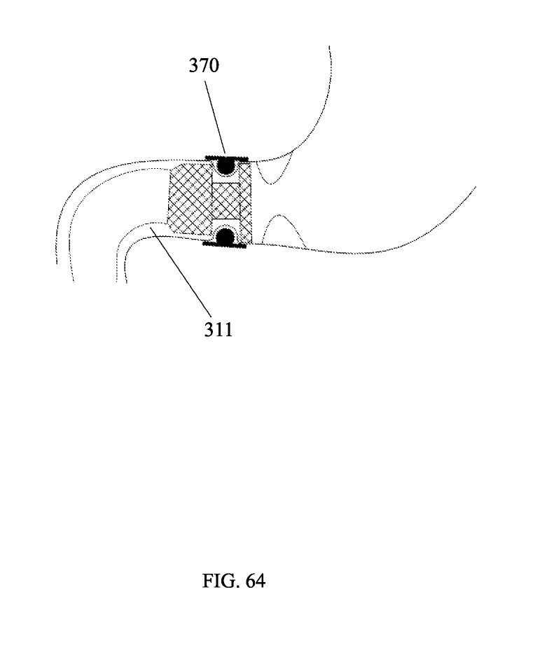

As a further illustration of the combinations possible, FIG. 64 shows a combination of an external band 370 of a different design interlocks with the internal implant design 311 with a proximal hour-glass shape end 320 as shown in FIG. 54.

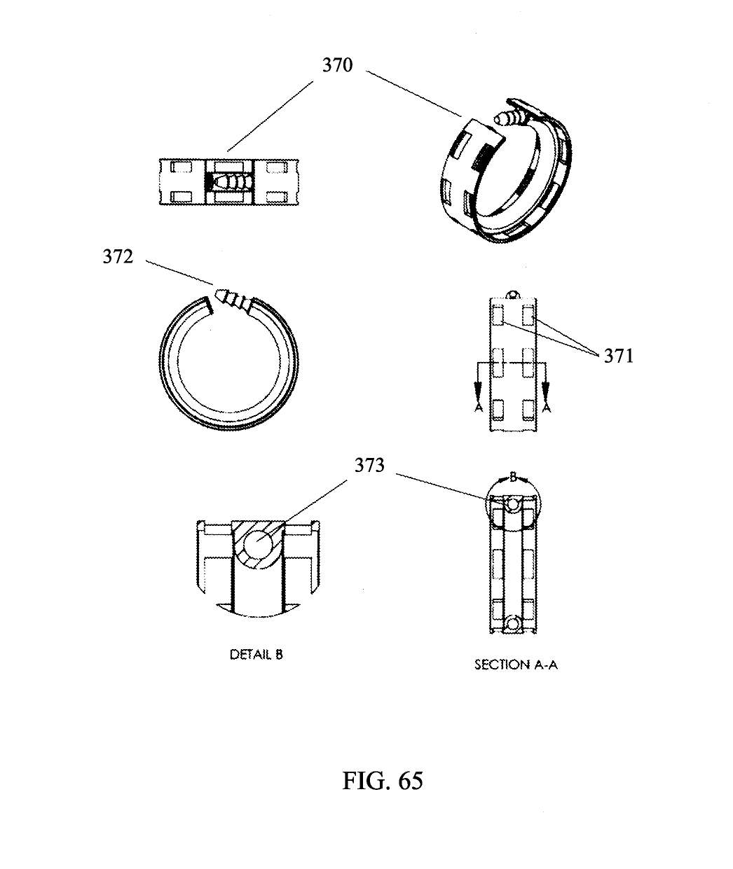

FIG. 65 shows the design of the external implant 370 in detail. External implant 370 consists of a central ring with a circular cross-section attached to a flat band made of compliant materials. The flat band has slots 371 on both sides of the tubular central ring and by forces exerted by both the top and the bottom flanges of the hour-glass-shaped implant, tissue will extrude through the slots providing for safe and secure interlocking of the implants. It also has a means to adjust the diameter of the implant and lock it around the duodenum by means of a barbed end 372 that can be inserted in to the circular aperture 373 at the other end of the band.

While the above described embodiments of the external and internal implants in combination are likely to hold the internal bypass sleeve elements securely within the small intestine it also possible to provide even more superior anchoring.

According to various embodiments, as a second mode of anchoring, stabilizing, or preventing migration, the external bands of design 310, 330, 340, 360 and 370 can be coupled to an anatomical feature (e.g., a ligament) external to the tissue of the stomach, pylorus, or intestine. In some embodiments, the external band 310 is intertwined with or threaded between the anatomical feature and the tissue. According to one exemplary embodiment, the external band 310 is coupled to the hepato-duodenal ligament which suspends and tethers the liver and the duodenum in the abdomen. These anatomical structures thus will provide positive spatial positioning of the interlocking internal and external implant pairs described before. This second mode of anchoring enables the use of external bands that do not rely on excessive compressive force to keep the implant in place, as excessive compressive forces can cause tissue necrosis and erosion.

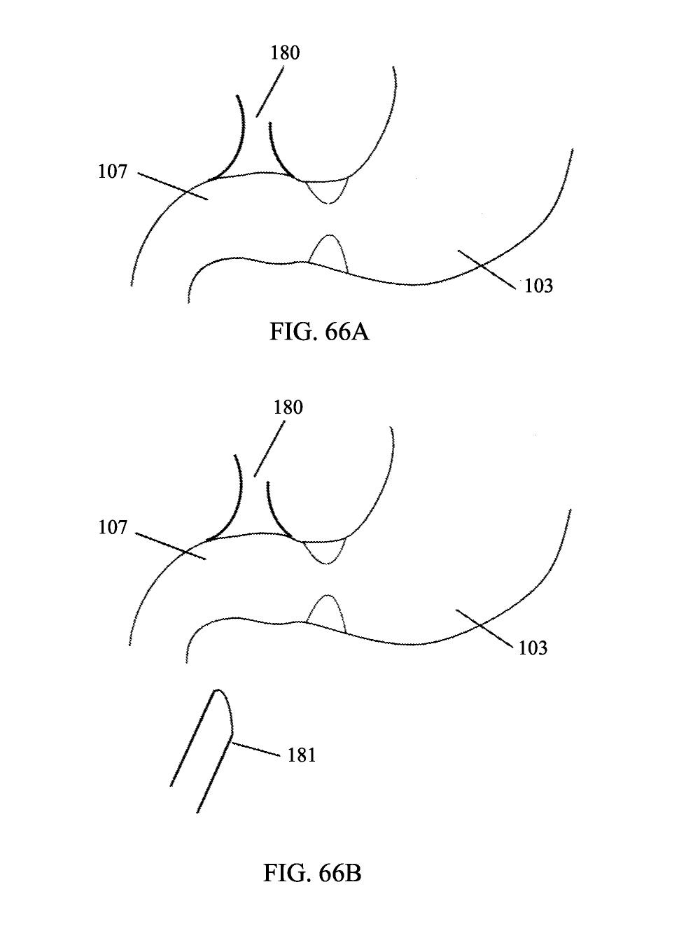

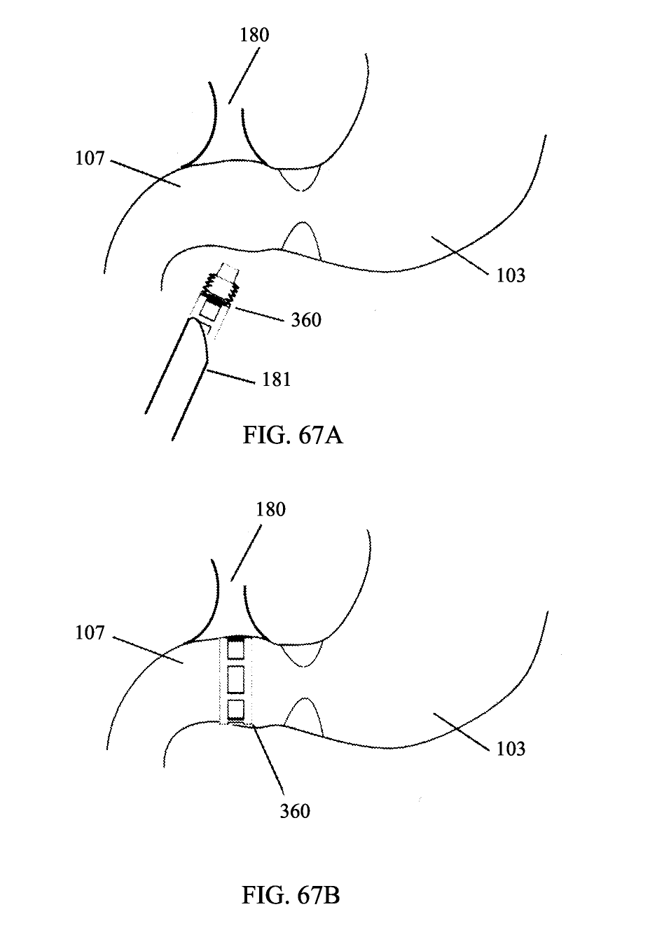

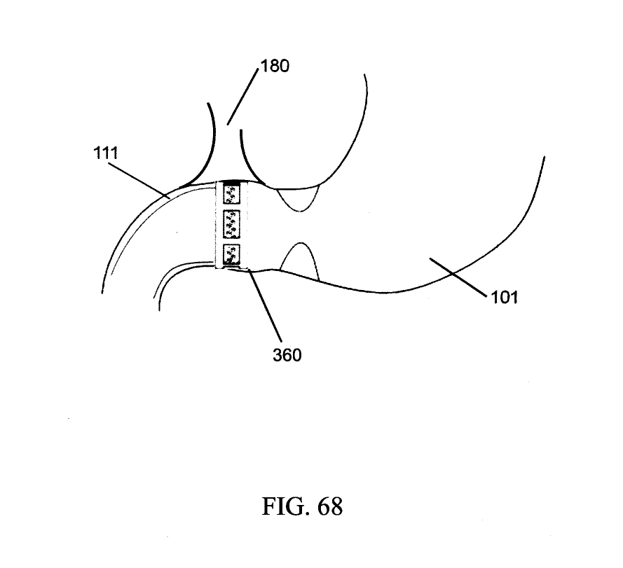

FIGS. 66A, 66B, 67A, 67B and 68 show the sequence of steps a physician would perform in order to place the combination of the external implant and the internal bypass sleeve implant in the body. The external implant would be placed using laparoscopic means as shown in steps 1 through 4. FIGS. 66A and 66B show the initial steps of the procedure in which laparoscopic access is achieved, the anatomy is visualized, and a trocar 181 for delivering the external implant 360 is inserted into the patient. FIGS. 67A and 67B show the steps needed to advance the external implant 360 and position it around the duodenal bulb 107. The external implant is preferably threaded through the hepato-duodenal ligament 180 to prevent migration of the device. Subsequently using endoscopic technique the internal implant 311 will be inserted from the mouth and the stomach in to the duodenum; the sleeve element will be first deployed and finally the proximal internal implant will be positioned to align with the external implant and deployed as shown in step 5 shown in FIG. 68. In order to align the external implant and the internal implants, radio-opaque marker bands might be placed at key locations on both the external and internal implants to ensure correct positioning and secure interlocking

In order to remove the internal implant a reverse procedure would be followed. In the spring type implant, the drawstring would be hooked in order to collapse the implant to a small diameter and then withdraw it orally.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

The following is claimed:

1. A gastrointestinal implant system for treating metabolic disorders such as diabetes and obesity, the system comprising:
a tubular implant adapted for placement within at least a portion of the duodenum, the tubular implant having a securing feature; and
an external band configured for implantation around at least one of a pylorus, and a duodenum, the external band having a coupling feature for removably engaging and coupling with the securing feature of the internal tubular implant, without penetrating the duodenum or pylorus, such that the therapeutic implant resists migration within the gastrointestinal tract;
wherein the securing feature and coupling feature are configured such that the tubular implant is releasably coupled to the external band to facilitate removal of the tubular implant.

2. The gastrointestinal implant system of claim 1 wherein the securing feature and the coupling feature are configured to secure the tubular implant to the external band by mechanical coupling.

3. The gastrointestinal implant system of claim 1 wherein a diameter of the coupling feature of the external band is mechanically adjustable.

4. The gastrointestinal implant system of claim 1 wherein a diameter of the coupling feature of the external band is adjustable using an inflatable member.

5. The gastrointestinal implant system of claim 1 wherein the tubular implant extends within the duodenum such that a distal end of the implant is located near or past the ligament of Treitz.

6. A modular gastrointestinal implant system for treating metabolic conditions such as diabetes and obesity, the system comprising:
an external implant configured for affixing around at least a portion of a duodenum or pylorus, the external implant having a docking feature; and
a therapeutic implant adapted for placement within a gastrointestinal tract, the therapeutic implant having a securing feature adapted to removably couple with the docking feature without penetrating the gastrointestinal tract, such that the therapeutic implant resists migration within the gastrointestinal tract;
wherein the external band has an implanted diameter slightly smaller than outer diameter of the duodenum or pylorus.

7. The modular implant system of claim 6 wherein the docking feature is a stent.

8. The modular system of claim 6 wherein the docking feature is a metal band.

9. The modular system of claim 6 wherein the docking feature is a fabric or elastomeric band.

10. The modular system of claim 6 wherein the securing feature and the docking feature are mechanical elements adapted to couple without penetrating the gastrointestinal tract.

11. The modular system of claim 6 wherein the therapeutic implant includes a long tubular element adapted to function as a conduit for food and organ secretions.

12. The modular system of claim 6 wherein the therapeutic implant has a length selected such that a distal end of the implant is located at or near the ligament of Treitz.

13. A method of treating metabolic conditions such as diabetes and obesity, the method comprising:
placing an external implant around at least a portion of the duodenum, the external implant having a docking feature and the external implant having an inner diameter slightly smaller than an outer diameter of the corresponding portion of the duodenum;
implanting, using a minimally-invasive technique, an internal tubular implant having a securing feature to a location within the duodenum corresponding to the location of the external implant; and
causing the securing feature to removably couple with the docking feature without penetrating the duodenum;

14. The method of claim 13 wherein the securing feature and the docking feature are mechanical element adapted to interlock without penetrating the gastrointestinal tract.

15. The method of claim 13 wherein the placing step includes interlocking the external band with an anatomical structure at or near the desired implant location along the duodenum.
